SEQUENCE LISTING (25 sequences)

SEQ ID NO: 1 - protein, 735 aa, Synthetic construct [sequence omitted]

SEQ ID NO: 2 - protein, 745 aa, Synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVTTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 3 - protein, 745 aa, Synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVTTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYFLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 4 - protein, 745 aa, Synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVTTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGEVTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 5 - protein, 745 aa, Synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVTTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGDVTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 6 - protein, 745 aa, Synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - DNA, 2238 bp, Synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcaccaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacctagcactcggcgaaacaacaagacctgctaggcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctattaatgtggactttactgtggacactaatggcgtgtattcagagcctcgccccattggcaccagatacctgactcgtaatctgtaa

SEQ ID NO: 8 - DNA, 2238 bp, Synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcaccaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtatttcttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacctagcactcggcgaaacaacaagacctgctaggcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctattaatgtggactttactgtggacactaatggcgtgtattcagagcctcgccccattggcaccagatacctgactcgtaatctgtaa

SEQ ID NO: 9 - DNA, 2238 bp, Synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcaccaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacctagcactcggcgaagtaacaagacctgctaggcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctattaatgtggactttactgtggacactaatggcgtgtattcagagcctcgccccattggcaccagatacctgactcgtaatctgtaa

SEQ ID NO: 10 - DNA, 2238 bp, Synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcaccaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacctagcactcggcgatgtaacaagacctgctaggcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctattaatgtggactttactgtggacactaatggcgtgtattcagagcctcgccccattggcaccagatacctgactcgtaatctgtaa

SEQ ID NO: 11 - DNA, 28 bp, Synthetic construct
  source 1..28: mol_type = other DNA
aacaataaatgatttaaatcaggtatgg

SEQ ID NO: 12 - DNA, 28 bp, Synthetic construct
  source 1..28: mol_type = other DNA
gtggtggtgactctgtcgcccatccatg

SEQ ID NO: 13 - DNA, 29 bp, Synthetic construct
  source 1..29: mol_type = other DNA
acagagtcaccaccaccagcacccgaacc

SEQ ID NO: 14 - DNA, 41 bp, Synthetic construct
  source 1..41: mol_type = other DNA
ttgttgtttcgccgagtgctaggttgcctctctggaggttg

SEQ ID NO: 15 - DNA, 42 bp, Synthetic construct
  source 1..42: mol_type = other DNA
tcggcgaaacaacaagacctgctaggcaagcagctaccgcag

SEQ ID NO: 16 - DNA, 28 bp, Synthetic construct
  source 1..28: mol_type = other DNA
acattaatagacttgttgtagttggaag

SEQ ID NO: 17 - DNA, 30 bp, Synthetic construct
  source 1..30: mol_type = other DNA
acaagtctattaatgtggactttactgtgg

SEQ ID NO: 18 - DNA, 24 bp, Synthetic construct
  source 1..24: mol_type = other DNA
gctgtttaaacgcccgggctgtag

SEQ ID NO: 19 - DNA, 28 bp, Synthetic construct
  source 1..28: mol_type = other DNA
gctcaagaaatacaggtactggtcgatg

SEQ ID NO: 20 - DNA, 29 bp, Synthetic construct
  source 1..29: mol_type = other DNA
cctgtatttcttgagcagaacaaacactc

SEQ ID NO: 21 - DNA, 29 bp, Synthetic construct
  source 1..29: mol_type = other DNA
gtcttgttacttcgccgagtgctaggttg

SEQ ID NO: 22 - DNA, 33 bp, Synthetic construct
  source 1..33: mol_type = other DNA
cactcggcgaagtaacaagacctgctaggcaag

SEQ ID NO: 23 - DNA, 29 bp, Synthetic construct
  source 1..29: mol_type = other DNA
tcttgttacatcgccgagtgctaggttgc

SEQ ID NO: 24 - DNA, 30 bp, Synthetic construct
  source 1..30: mol_type = other DNA
tcggcgatgtaacaagacctgctaggcaag

SEQ ID NO: 25 - DNA, 6148 bp, Synthetic construct
  source 1..6148: mol_type = other DNA
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatatcaagcttcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggatatcggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggcccagccggcctcgcgagaattctctagaacgccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagagctgacactagtgcggatccacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

(19) United States
(12) Patent Application Publication
(10) Pub. No.: US 2026/0041795 A1
(43) Pub. Date: Feb. 12, 2026

(54) ADENO-ASSOCIATED VIRUS CAPSID HAVING TISSUE TROPISM AND USE THEREOF

(71) Applicant: SHANGHAI LANGSHENG BIOTECHNOLOGY CO., LTD, Shanghai (CN)

(72) Inventors: Fei WU, Shanghai (CN); Zhuoyu NI, Shanghai (CN); Fenghua WANG, Shanghai (CN)

(21) Appl. No.: 19/152,333

(22) PCT Filed: Jan. 16, 2024
(86) PCT No.: PCT/CN2024/072554

§ 371 Date: Jul. 30, 2025

(30) Foreign Application Priority Data
Jan. 31, 2023 (CN) 202310084749.8

(51) Int. Cl. A61K 48/00; A61K 45/00; C12N 15/86

(52) U.S. Cl. CPC A61K 48/0058; A61K 45/05; A61K 48/0075; C12N 15/86; C12N 2750/14122; C12N 2750/14143; C12N 2750/14152

(57) ABSTRACT

An adeno-associated virus (AAV) capsid protein, a recombinant AAV virion containing same, and the use thereof in the treatment and prevention of diseases.

### TECHNICAL FIELD

The present invention relates to an adeno-associated virus (AAV) capsid protein, a recombinant AAV viral particle containing the capsid protein and use thereof in the treatment and prevention of diseases.

### BACKGROUND OF THE INVENTION

Adeno-associated virus (AAV) belongs to the family Parvoviridae, and the genus Dependovirus, members of which need to be co-infected with a helper virus such as adenovirus to initiate replication, and AAV establishes a latent infection in the absence of a helper virus. The viral particle consists of an icosahedral shell encompassing a 4.9 kb single-stranded DNA genome with two open reading frames: rep and cap. The nonstructural rep gene encodes four regulatory proteins necessary for viral replication, while the cap encodes three structural proteins assembled into a 60-mer capsid shell, i.e., capsid proteins VP1, VP2, and VP3.

Adeno-associated virus (AAV) has characteristics of low pathogenicity and being able to stably express proteins in various organs and tissues for a long time, by which AAV has an obvious advantage in the field of gene therapy and are suitable for delivering therapeutic genes. However, wild-type AAV serotypes usually infect multiple tissues or organs of mammals broadly and have a wide range of tissue targeting, resulting in gene delivery to off-target tissues, thereby exacerbating adverse reactions. The capsid protein of AAV viral particles not only regulates the assembly of AAV during replication, but also promotes the interaction between the virus and receptors on the plasma membrane and entry into target cells.

Studies have shown that the tissue tropism and cell transformation efficiency of AAV vectors are mainly determined by their capsids. In view of this, in order to improve the treatment effect of ophthalmological related genetic diseases, it is expected to carry out directed and rationally designed engineering of AAV capsid proteins to obtain organ-specific (especially eye-specific) AAV vectors.

It is known that all of AAV types 1, 2, 4, 5, 7, 8 and 9 can transduce retinal pigment epithelial cells or photoreceptor cells through intraretinal or local administration. However, the transduction efficiency of the serotypes in the above-mentioned prior art will be greatly reduced via IVT (intravitreal) administration. The known serotype of AAV2.7m8 involved in the existing patent technology (CN103561774B) is obtained by engineering the AAV2 virus based on the following principles: the integration of an exogenous 7-amino acid short peptide (which can significantly enhance the penetration of AAV2 through the inner limiting membrane and reach the retinal tissue) at the 587 and 588 amino acid positions of the AAV2 capsid VP1 can change the ability of the AAV2 virus capsid to bind to the HSPG receptor. However, intravitreal administration of AAV2.7m8 cannot effectively transduce the outer retinal tissues (especially the retinal pigment epithelium and photoreceptor cell layer). The existing serotypes can only infect the inner retinal tissues in large animals and cannot penetrate to arrive the photoreceptor cell layer (outer retina).

Therefore, there is an urgent need for a new tissue-tropic recombinant AAV viral particle with higher in vitro and in vivo transduction activity against ocular tissues, especially retinal tissues, which can, for example, effectively penetrate the layer of inner segment and layer of PR of the retinal tissue under IVT administration, and effectively reach the layer of RPE, thus achieving full-layer distribution in the retinal tissue.

### SUMMARY OF THE INVENTION

The tissue specificity (i.e., tissue tropism) of AAV is determined by the serotype of capsid, and the rationally designed new serotype of the AAV capsid according to the present invention can alter the tissue tropism of conventional AAV while improving the tissue transduction activity of AAV. Specific amino acid point mutation modifications on the capsid surface and the insertion of exogenous peptides can affect the specificity and transduction activity of AAV delivery therapies. For example, new design considerations of altering the receptor-recognizing motif of AAV capsids, deleting and additionally adding the receptor binding sites on the capsid surface may be beneficial for improving the transduction efficiency of AAV in retinal tissues. For example, when eye tissue is the desired target, i.e., the tissue tropism for natural eyes can be enhanced, or when eye tissue is not the desired target, the tissue tropism of the natural AAV can be reduced. In clinical application scenarios, when it is desired that the virus can more effectively deliver the target gene to different cells in the retina of the eyeball and transduce them more effectively, a low dose of the new AAV serotype (e.g., the new serotype RC-C14/RC-C02 of the present invention) can be administered to the subject.

It is known that the main receptor for capsid of serotype AAV2 to enter cells is heparan sulfate glycoprotein (HSPG), and it can also enter cells with the help of accessory receptors such as integrin, FGFR, HGFR, LamR, AAVR, etc (the efficiency mediated by auxiliary receptor is low). The core of the invention is to insert a short amino acid peptide into the AAV capsid protein and mutate the specific site based on rational design. Therefore, RC-C02 (2 point mutations I240T-V708I) and RC-C03 (3 point mutations I240T-V708I-Y444F) were rationally designed based on AAV2.7m8 (RC-C01): further, through computer simulation of tissue-targeted short peptides, new serotypes such as RC-C13 and RC-C14 were screened out, in which 10 amino acids were inserted after position 587 of VP1 (587-LALGEVTRPA or 587-LALGDVTRPA). Through the analysis of the three-dimensional steric structure of the RC-C14 capsid, it was found that: the integration of 10-mer peptides in the VP1 VIII variable region can introduce a new targeting short peptide on the surface of the AAV capsid protein, thus changing the steric structure for the original receptor binding, resulting in the generation on the surface of AAV capsid of a switch on specifically binding to potential new receptors, while weakening the binding activity of rAAV2 with HSPG. The biological properties of the new serotype (RC-C14) include the ability to efficiently enter photoreceptors and melanocytes by binding to multiple receptors, including the primary binding receptor for entering cells by binding to the heparin recognition receptor HSPG, resulting in a significant increase in transduction efficiency in cell lines in vitro and in retinal tissue in vivo.

Accordingly, in one aspect, the present invention relates to a method of obtaining VP1 variants of a recombinant AAV viral particle (rAAV) having tissue tropism;
(1) Based on AAV2 capsid protein VP1, through three-dimensional steric structure analysis of the capsid protein, potential receptor recognition positions exposed on the capsid surface is searched, and a directional rational design for synthesis of the AAV2 serotype capsid VP1 was conducted. A proper integration position is found on capsid protein surface, into which a 5mer-15mer, such as 6mer, 7mer, 8mer or 9mer, 10mer functional short peptide is inserted, and a new HSPG receptor-independent serotype is screened; and/or
(2) Since tyrosine residues on the surface of the rAAV capsid are easily recognized by protease and further influence the transduction activity, amino acids at specific exposed positions on the surface of the capsid are mutated (for example, RC-C14 serotype is obtained) in the present invention, thereby improving the stability of the virus in cells, maintaining the stability of the capsid, and significantly improving the transduction efficiency to retinal cells (the transduction efficiency to retinal tissues in vivo is significantly improved compared with the existing serotypes).

The new serotype (e.g., new serotype RC-C14) rAAV and variants derived therefrom obtained by the present invention include one or more of the following surprising improvements;
(i) The structure of capsid is more stable;
(ii) The viral production packaged under the same conditions is higher, and the empty capsid ratio is lower than that of AAV2;
(iii) The transduction activity to photoreceptor cells (PR) and/or retinal pigment epithelial cells (PRE) is higher than that of known serotypes. For example, compared with the AAV2.7m8 serotype, the transduction activity of the new serotype administered intravitreally to PR (in mice and cynomolgus monkeys) is significantly improved;
(iv) The clinical application value and the commercialization potential are huge.

The new serotype (e.g., new serotype RC-C14) rAAV and variants derived therefrom obtained by the present invention further include one or more of the following surprising improvements: the new serotype (e.g., RC-C14 serotype) of the present invention has significantly enhanced tropism for retinal cells (PR and RPE) compared with existing serotypes. At the same time, the chimerism of 10-mer peptide (e.g., LALGDVTRPA) on the capsid surface (VP1 586-590aa) significantly weakens the ability of the AAV2 serotype capsid to bind to HSPG (primary receptor), while ensuring efficient inner limiting membrane penetrating ability. Compared with existing serotypes, the new serotype of the present invention has further enhanced tropism for photoreceptor cells. Whether it is administered subretinally or intravitrealally, the new serotype of the present invention can be distributed in all layers of the retina, stably and continuously express exogenous supplementary proteins, and the stability of the virus is better than that of existing known serotypes.

The present invention therefore relates to the following specific embodiments:

1. An AAV capsid protein variant comprising an engineered capsid protein VP1, the VP1 comprising amino acid substitutions I240T and V708I relative to a parental AAV capsid protein VP1, wherein the amino acid positions are determined with reference to the amino acid sequence position of SEQ ID NO: 1.

2. The AAV capsid protein variant of embodiment 1, further comprising a Y444F substitution and/or a T491V substitution.

3. The AAV capsid protein variant of embodiment 1 or 2, further comprising an insertion of 5-15 amino acids, e.g., 5-10 amino acids, preferably 10 amino acids, between positions 587 and 588.

4. The AAV capsid protein variant of embodiment 3, wherein fragments LALGETTRPA, LALGDVTRPA, or LALGEVTRPA is inserted between positions 587 and 588.

5. The AAV capsid protein variant of any one of embodiments 1 to 4, comprising, or consisting of only, the following amino acid mutations;
(1) I240T-V708I and an insertion of a fragment LALGETTRPA, LALGDVTRPA, or LALGEVTRPA between positions 587-588;
(2) I240T-V708I-Y444F and an insertion of a fragment LALGETTRPA, LALGDVTRPA, or LALGEVTRPA between positions 587-588;
(3) I240T-V708I-Y444F-T491V and an insertion of the fragments LALGETTRPA, LALGDVTRPA, or LALGEVTRPA between positions 587-588.

6. The AAV capsid protein variant of embodiment 1 or 2, wherein the parental AAV capsid protein VP1 is from AAV serotype 2 (AAV2) or AAV2 variant version 7m8 (AAV2.7m8).

7. The AAV capsid protein variant of any one of embodiments 3 to 5, wherein the parental AAV capsid protein VP1 is from AAV serotype 2 (AAV2).

8. The AAV capsid protein variant of any one of embodiments 1 to 7, wherein the AAV capsid protein variant
(i) comprises the amino acid sequence as set forth in SEQ ID NO:2; or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:2, and comprises the substitutions I240T-V708I and the insert 587-LALGETTRPA-588;
(ii) comprises the amino acid sequence as set forth in SEQ ID NO:3: or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:3, and comprises the substitutions I240T-V708I-Y444F and the insert 587-LALGETTRPA-588;
(iii) comprises the amino acid sequence as set forth in SEQ ID NO:4; or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:4, and comprises the substitutions I240T-V708I and the insert 587-LALGEVTRPA-588; or
(iv) comprises the amino acid sequence as set forth in SEQ ID NO:5: or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:5, and comprises the substitutions I240T-V708I and the insert 587-LALGDVTRPA-588.

9. The capsid protein variant of any one of embodiments 1 to 7, which is
(i) encoded by the nucleic acid sequence as set forth in SEQ ID NO:7; or encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:7 and comprises the substitution I240T-V708I and the insert 587-LALGETTRPA-588;
(ii) encoded by the nucleic acid sequence as set forth in SEQ ID NO:8: or encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:8 and comprises the substitution I240T-V708I-Y444F and the insert 587-LALGETTRPA-588;
(iii) encoded by the nucleic acid sequence as set forth in SEQ ID NO:9; or encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:9 and comprises the substitution I240T-V708I and the insert 587-LALGEVTRPA-588; or
(iv) encoded by the nucleic acid sequence as set forth in SEQ ID NO:10; or encoded by a nucleic acid sequence having at least 85%, 86%, 87%, 88%, 89%, 90%, 91%, 92%, 93%, 94%, 95%, 96%, 97%, 98% or 99% identity to the nucleic acid sequence as set forth in SEQ ID NO:10 and comprises the substitution I240T-V708I and the insert 587-LALGDVTRPA-588.

10. An isolated nucleic acid comprising a nucleotide sequence encoding the AAV capsid protein variant according to any one of embodiments 1-9.

11. The isolated nucleic acid of embodiment 10, wherein the nucleotide sequence encodes a capsid protein, which
(i) comprises or consists of the nucleic acid sequence as set forth in SEQ ID NO:7; or comprises a nucleic acid sequence having at least 85%, 86%, 87%, 88%, 89%, 90%, 91%, 92%, 93%, 94%, 95%, 96%, 97%, 98% or 99% identity to the nucleic acid sequence as set forth in SEQ ID NO: 7, and the capsid protein variant encoded by said nucleic acid sequence comprises the substitution I240T-V708I and the insert 587-LALGETTRPA-588;
(ii) comprises or consists of the nucleic acid sequence as set forth in SEQ ID NO:8: or comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO: 8, and the capsid protein variant encoded by said nucleic acid sequence comprises the substitution I240T-V708I-Y444F and the insert 587-LALGETTRPA-588;
(iii) comprises or consists of the nucleic acid sequence as set forth in SEQ ID NO:9; or comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO: 9, and the capsid protein variant encoded by said nucleic acid sequence comprises the substitution I240T-V708I and the insert 587-LALGEVTRPA-588;
(iv) comprises or consists of the nucleic acid sequence as set forth in SEQ ID NO:10; or comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO: 10, and the capsid protein variant encoded by said nucleic acid sequence comprises the substitution I240T-V708I and the insert 587-LALGDVTRPA-588.

12. A recombinant AAV viral particle (rAAV) comprising
(i) the AAV capsid protein variant of any one of embodiments 1 to 9; and optionally
(ii) a nucleic acid of interest encoding, e.g., a prophylactic or therapeutic protein, packaged within the AAV capsid, e.g., the nucleic acid of interest is selected from an ophthalmic related gene, e.g., RPE65, AIPL1, PROM1, or RS1.

13. The recombinant AAV viral particle of embodiment 12, wherein the nucleic acid of interest is comprised in an expression cassette and thus packaged within the AAV capsid.

14. The recombinant AAV viral particle of embodiment 13, wherein the expression cassette is single-stranded DNA, double-stranded DNA or single-stranded RNA or double-stranded RNA.

15. A method of producing a recombinant AAV viral particle, comprising culturing a packaging cell under conditions sufficient for production of a recombinant AAV viral particle, wherein the packaging cell comprises a plasmid comprising a nucleic acid encoding the capsid protein variant of any one of embodiments 1 to 9, or the nucleic acid of embodiment 10 or 11.

16. The method according to embodiment 15, wherein the packaging cell further comprises a helper plasmid and/or a transfer plasmid comprising the nucleic acid of interest.

17. The method of embodiment 15 or 16, further comprising isolating from the culture supernatant a self-complementary recombinant adeno-associated virus (rcAAV) particle.

18. The method of any one of embodiments 15 to 17, further comprising lysing the packaging cell, and isolating a recombinant AAV viral particle from the cell lysate.

19. The method of any one of embodiments 15-18, further comprising one or more of the following steps;
a. removing the cell debris,
b. treating the supernatant containing the recombinant AAV viral particle with a benzonase nuclease,
c. concentrating the recombinant AAV viral particle, and
d. purifying the recombinant AAV viral particle.

20. A recombinant AAV viral particle prepared according to the method of any one of embodiments 15-19.

21. A plasmid, such as an expression plasmid, comprising a nucleic acid encoding the capsid protein variant according to any one of embodiments 1 to 9 or a nucleic acid according to embodiment 10 or 11.

22. A packaging cell for producing a recombinant AAV viral particle, the packaging cell comprising a plasmid comprising a nucleic acid encoding the capsid protein variant of any one of embodiments 1-9 or the nucleic acid of embodiment 10 or 11.

23. A formulation or composition or medicament comprising the recombinant AAV viral particle of any one of embodiments 12-14 or 20, and optionally pharmaceutically acceptable auxiliary materials, such as pharmaceutically acceptable carriers, excipients, including buffers, as known in the art.

24. A combination product comprising the recombinant AAV viral particle according to any of embodiments 12-14 or 20, and one or more additional therapeutic agents, e.g., an immunomodulator, e.g., immunosuppressant.

25. A method of treating an ocular disease in an individual comprising administering to the individual the recombinant AAV viral particle of any one of embodiments 12-14 or 20 or the formulation or composition of embodiment 23 or the combination product of embodiment 24.

26. The method according to embodiment 25, wherein the administration can be an intraocular administration, e.g., an intraretinal administration or intravitreal administration, e.g., a subretinal administration or intravitreal administration.

27. The method according to embodiment 26, wherein the administration is injection.

### BRIEF DESCRIPTION OF THE DRAWINGS

FIG. 1: Construction of capsid plasmids of four mutant serotype and virus preparation. Wherein FIG. 1A shows a plasmid map of capsids of four serotype; FIGS. 1B-1C show the viral yields of the four rAAV species in the cells and supernatant, respectively.

FIG. 2: Comparison of in vitro transduction activity of AAV2 serotypes and variants thereof. Wherein FIG. 2A shows a picture of green fluorescence after in vitro infection of HEK293T with AAV2 and variants thereof at two MOIs; FIG. 2B shows a fluorescent photograph of four rAAVs in APRE19; FIGS. 2C-2F represent the fluorescence ratio statistics (FACS) of the four serotypes mentioned above in vitro transduction into 293T, ARPE19, 661W, CHO, respectively, and Mock represents a blank control.

FIG. 3: Comparison of transduction activity of AAV2 serotypes and variants thereof in various cell lines. FIGS. 3A-3D show the green fluorescence ratio and Mean Fluorescence Intensity (MFI) of AAV2 serotypes and variants thereof in CHO and ARPE19, respectively; FIGS. 3E and F show the green fluorescence ratio (FIG. 3E) and Mean Fluorescence Intensity (MFI) (FIG. 3F) of AAV2 serotype variants after in vitro infection of 661W at two MOIs, respectively; FIG. 3G shows a comparison of the infection activity of the four serotype variants in HEK293T cells (ratio of infected cells); FIG. 3H shows the information of mutation site in capsid in different serotype plasmids: the specific information is as follows: RC-C01 is AAV2.7m8; RC-C02 has mutations I240T and V708I relative to AAV2.7m8, i.e., has an insert of 587-LALGETTRPA-588 relative to AAV2, as well as I240T and V708I; RC-C03 has mutations I240T, Y444F and V708I relative to AAV2.7m8, i.e., has an insert of 587-LALGETTRPA-588 relative to AAV2, and I240T, Y444F and V708I; RC-C13 has an insert of 587-LALGEVTRPA-588 relative to AAV2, as well as I240T and V708I; and RC-C14 has an insert of 587-LALGDVTRPA-588 relative to AAV2, as well as I240T and V708I.

FIG. 4: Comparison of the in vivo transduction activity of RC-C02 and RC-C14 serotypes to existing serotypes (AAV2 and AAV2. GL). Wherein FIG. 4A shows a photograph of BAF fluorescence in vivo imaging of mice after 2 and 4 weeks of administration; FIG. 4B shows a statistical graph of fluorescence area; FIG. 4C shows a photograph of panoramic and localized fluorescent staining of retinal tissue sections following IVT administration of AAV (wherein the nucleus is labeled by DAPI, EGFP-green autofluorescence protein): FIG. 4D shows a photograph of panoramic and localized fluorescent staining of retinal tissue sections following subretinal administration of AAV.

FIG. 5: Comparison of tissue distribution and differences in transduction efficiency of RC-C14 and variants thereof after IVT administration. Wherein FIG. 5A shows a photograph of a mouse eye tissue in vivo fluorescence imaging (IVT administration); FIG. 5B shows a statistical graph of the fluorescence area of FIG. 5A.

FIG. 6: Study on the distribution of the two serotypes RC-C14 and RC-C02 in mouse retinal tissue. FIG. 6A shows a fluorescent photograph of retinal tissue of mice infected with three serotypes 4 weeks after IVT administration; FIG. 6B shows a fluorescent photograph of the retinal tissue infected with the three serotypes (4 weeks after SR (subretinal) administration).

FIG. 7: Study on the distribution of the two serotypes RC-C14 and RC-C02 in NHP retinal tissue. Wherein FIG. 7A shows a fundus autofluorescence photograph (BAF) of monkey eyes 4W after administration of two serotypes, and FIG. 7B shows an immunofluorescence labeling photograph of retinal tissue sections of monkey eyes (RPE cells are labeled with RPE65 red-labeled antibody, PR photoreceptor cells are labeled with Opsin red-labeled antibody, M is the abbreviation of Merge, representing a combination of red and green pictures).

FIG. 8: Comparison of transduction activities of RC-C14 and its 6 variants in different cell lines. Wherein FIGS. 8A-8B show statistical analysis of the infection efficiency and transduction intensity of different serotypes in 293T; FIGS. 8C-8D show statistical analysis of the transduction efficiency and transduction intensity of different serotypes in ARPE19; FIGS. 8E-8F show statistical analysis of transduction efficiency and intensity of different serotypes in 661W; and FIG. 8G shows the detailed mutation information for capsid VP1 amino acids of six variants of RC-C14.

### DETAILED DESCRIPTION OF THE INVENTION

### I. Definitions

For purposes of interpreting this specification, the following definitions will be used, and where appropriate, terms used in the singular may also include the plural, and vice versa. Unless otherwise specified, all technical and scientific terms used herein have the same meanings as those generally understood by a person of ordinary skill in the art to which the present invention belongs.

The term “about” or “approximately” is included within a statistically significant range of values. Such ranges may be within an order of magnitude of a given value or range, preferably within 50%, more preferably within 20%, still more preferably within 10%, and even more preferably within 5% or within 1%. The allowable variations covered by the terms “about” or “approximately” depend on the particular system under study and can be readily appreciated by one of ordinary skill in the art.

As used herein, the term “and/or” means any one of the options or two or more or all of the options.

As used herein, the terms “comprising” or “including” means including the recited elements, integers, or steps, but not excluding any other elements, integers, or steps. When the term “comprising” or “including” is used herein, unless otherwise indicated, it also encompasses the situation consisting of the stated elements, integers, or steps. For example, reference to an antibody variable region “comprising” a particular sequence is also intended to encompass antibody variable regions consisting of that particular sequence.

As used herein, adeno-associated virus (AAV), also known as adeno-associated virus, belongs to the genus Dependovirus in the family Parvoviridae, and is a single-stranded DNA-deficient virus with the simplest structure that is currently discovered, requiring a helper virus (usually an adenovirus) to participate in replication. It encodes the cap gene and the rep gene in inverted repeats (ITRs) at both ends. ITRs are crucial for the replication and packaging of viruses. The cap gene encodes the capsid protein of the virus, and the rep gene is involved in the replication and integration of the virus. AAV can infect a variety of cells. As the adeno-associated virus is smaller than other virus vectors and has no pathogenicity, and can transfect dividing and non-dividing cells, the gene therapy method based on the AAV vector for ocular diseases, especially for inherited retinal degeneration, has attracted extensive attention. The recombinant adeno-associated virus (rAAV) is derived from non-pathogenic wild type adeno-associated virus, is considered to be one of the most promising gene transfer vectors due to the characteristics of good safety, wide host cell range (dividing and non-dividing cells), low immunogenicity, long time for expressing foreign genes in vivo and the like, and is widely applied to gene therapy and vaccine research in the world. Over 10 years of research, the biological properties of recombinant adeno-associated virus (rAAV) have been well understood, and many data have been accumulated on the effects of rAAV in various cells, tissues, and in vivo experiments. In medical research, rAAV is used in the study of gene therapy for a variety of diseases (including in vivo, in vitro experiments): meanwhile, the gene transfer vector is used as a characteristic gene transfer vector and is widely applied to the aspects such as gene function research, disease model construction, and gene knock-out mouse preparation.

The term “capsid protein” includes proteins that are part of the viral capsid. For adeno-associated viruses, the capsid proteins are commonly referred to as VP1, VP2, and/or VP3, and are each encoded by a single cap gene. For AAV, these three AAV capsid proteins are produced in an overlapping fashion from the cap Open Reading Frame (ORF) by using alternative mRNA splicing and/or alternative translation start codon usage, although all three proteins use a common stop codon. Warrington et. al (2004) J. virol. 78:6595 is incorporated herein by reference in its entirety. VP1 of AAV2 is generally translated from the ATG start codon (amino acid M1) on 2.4-kb mRNA, while VP2 and VP3 of AAV2 originate from the smaller 2.3-kb mRNA, using the weaker ACG start codon for VP2 (amino acid T138) and read-through translation to the next available ATG codon (amino acid M203) for the most abundant capsid protein VP3. The amino acid sequence of the capsid proteins of adeno-associated viruses is well known in the art and is generally conserved, in particular depending on parvovirus. See Rutledge et al., (1998) J. Virol. 72:309-19. Accordingly, although the amino acid positions provided herein may be provided with respect to the VP1 capsid protein of AAV, and as not specifically indicated, the amino acid positions provided herein are determined with reference to the amino acid position of AAV2 VP1 as set forth in SEQ ID NO:1. Skilled person will be able to determine individually and readily the position of the same amino acid within the VP2 and/or VP3 capsid protein of AAV, and the corresponding position of the amino acid in the different serotypes. The AAV capsid proteins described herein encompass AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), or AAV2.GL, and the like.

The term “rcAAV” or “rAAV” as used herein refers to a recombinant adeno-associated virus, also known as a recombinant adeno-associated viral particle or a recombinant AAV.

The term “retinal cell” as used herein may refer to any cell type, including the retina, such as retinal ganglion cells, amacrine cells, horizontal cells, bipolar cells, and photoreceptor cells (including rod and cone cells), muller glia cells, and retinal pigment epithelium cells.

As used herein, the phrase “operably linked” includes physical juxtaposition of components or elements (e.g., in three-dimensional space) that directly or indirectly interact with each other, or otherwise coordinate with each other to participate in a biological event, the juxtaposition achieving or allowing such interaction and/or coordination. In some embodiments, “operably linked” refers to the covalent attachment of related components or elements to each other. However, those skilled in the art will appreciate that in some embodiments, covalent attachment is not required to achieve effective operable linkage.

The term “capsid protein variant” includes the capsid proteins having at least one mutation (e.g., substitution, deletion or insertion) as compared to the corresponding capsid protein as a parent.

Herein, the amino acid mutation may be an amino acid substitution, deletion, or insertion. Any combination of substitutions, deletions or insertions may be made to achieve an optimized variant with the desired properties. Amino acid deletions and insertions include those at the amino and/or carboxy terminus of the polypeptide sequence, as well as those within the polypeptide sequence. In some embodiments, the amino acid mutation is an amino acid substitution, such as a single amino acid substitution, or a combination of several amino acid substitutions. In some embodiments, the amino acid mutation is an insertion, such as an insertion of several amino acid fragments.

When referring to the amino acid position of the capsid protein to be mutated herein, it is determined by reference to the amino acid sequence as set forth in SEQ ID NO:1. The corresponding amino acid positions on the hybrid proteins or polypeptides having additional amino acid sequences can be identified by amino acid sequence alignment with SEQ ID NO:1. For example, when referring to “1240”, it refers to isoleucine I at position 240 of SEQ ID NO:1, or the amino acid residue at the corresponding position aligned on the amino acid sequence of other capsid proteins.

In reference herein to mutations to the capsid protein, single amino acid substitutions are described in the following manner: [original amino acid residue/position/substituted amino acid residue] or [position/substituted amino acid residue]. For example, isoleucine (or other corresponding amino acid) at position 240 is substituted with threonine, which can be represented as I240T or as 240T. Accordingly, individual single amino acid substitutions can be linked by “and” or “-” to indicate combinatorial mutations at multiple given positions. For example, the combinatorial mutation of I240T and V708I can be expressed as: I240T-V708I.

In reference herein to mutations to the capsid protein, the insertion is described in the following manner: [position of original amino acid-insert-original amino acid position+1]. For example, the insert LALGDVTRPA at positions 587 and 588 may be represented as 587-LALGDVTRPA-588.

The terms “transduction” or “infection” and the like refer to the introduction of a nucleic acid into a target cell by a viral vector. The term “transduction efficiency” refers to the fraction (e.g., percentage) of cells that express the nucleotide of interest after incubation with a set number of viral vectors containing the nucleotide of interest. Well-known methods for determining transduction efficiency include fluorescence activated cell sorting of cells transduced with a fluorescent reporter gene, PCR for expression of the nucleotide of interest, and the like.

“Percent (%) identity” of an amino acid sequence or nucleic acid sequence refers to the percentage of amino acid residues/nucleotides in the candidate sequence that are identical to the amino acid residues/nucleotides of the specific sequence shown in the specification, after aligning the candidate sequence with the specific sequence shown in the specification and introducing gaps, if necessary, to achieve the maximum percent sequence identity, and not considering any conservative substitutions as part of the sequence identity. In some embodiments, the present invention contemplates variants of a protein or polypeptide or nucleic acid of the present invention that have a substantial degree of identity, e.g., at least 80%, 85%, 90%, 95%, 97%, 98%, or 99% or more identity, relative to the polypeptide or protein or nucleic acid specifically disclosed herein. The variant may comprise conservative changes.

The terms “individual” or “subject” are used interchangeably herein to refer to mammals. Mammals include, but are not limited to, domesticated animals (e.g., cows, sheep, cats, dogs, and horses), primates (e.g., humans and non-human primates such as monkeys), rabbits, and rodents (e.g., mice and rats). In particular, the individual is a human.

The term “treating” includes administering the composition or hybrid polypeptide to prevent or delay the onset of the symptoms, complications, or biochemical indicators of a disease, alleviate the symptoms, or arrest or inhibit the further development of the disease, condition, or disorder.

The term “preventing” includes the inhibition of the occurrence or development of a disease or disorder or the symptoms of a particular disease or disorder.

The term “pharmaceutically acceptable auxiliary material” refers to a diluent, adjuvant (e.g., Freund's adjuvant (complete and incomplete)), excipient, carrier or stabilizer, etc., with which the active substance is administered.

The term “pharmaceutical composition” refers to a composition that is present in a form that allows the biological activity of the active ingredients contained therein to be effective, and which does not contain additional ingredients having unacceptable toxicity to the subject to which the composition is administered.

The term “effective amount” refers to an amount or dose of rAAV or composition or combination of the present invention which, upon administration to a patient in a single or multiple doses, produces the desired effect in the patient in need of treatment or prevention.

A “therapeutically effective amount” refers to an amount effective, at dosages and for periods of time required, to achieve the desired therapeutic result. A therapeutically effective amount is also one in which any toxic or adverse effect of the rAAV or composition or combination is less than a therapeutically beneficial effect. A “therapeutically effective amount” preferably inhibits or improve a measurable parameter by at least about 40%, even more preferably at least about 50%, 55%, 60%, 65%, 70%, 75%, 80%, 85%, 90% or even 100% relative to untreated subjects.

A “prophylactically effective amount” refers to an amount effective, at dosages and for periods of time required, to achieve the desired prophylactic result. Generally, a prophylactically effective amount will be less than a therapeutically effective amount because a prophylactic dose is used in a subject prior to or at an earlier stage of the disease.

The term “polynucleotide” refers to a polymeric form of nucleotides of any length, including deoxyribonucleotides or ribonucleotides or analogs thereof. A polynucleotide may comprise modified nucleotides, such as methylated nucleotides and nucleotide analogs, and may be interrupted by non-nucleotide components. As used herein, the term polynucleotide may refer to double-stranded and single-stranded molecules interchangeably.

The term “nucleic acid of interest” refers to a nucleic acid to be transduced by recombinant AAV viral particles, which encodes, for example, a prophylactic or therapeutic protein, in particular a protein for the prevention or treatment of ophthalmic diseases, such as AIPL1, PROM1, RS1 or antibody analogs etc.

### II. AAV Capsid Protein Variant

In some embodiments, the present invention relates to a new AAV capsid protein variant having one or more of the following properties;
(1) The variant is an HSPG receptor-independent serotype, i.e., has a weaker ability to bind to HSPG receptors but guarantees a highly efficient inner limiting membrane penetrating ability;
(2) The variant improves the stability of the virus in the cell;
(3) The structure of capsid is more stable;
(4) Compared with the existing serotype, the viral production packaged with the variant is higher and/or the empty capsid ratio is lower;
(5) The recombinant viral particles produced by the variant have significantly improved transduction efficiency (e.g., in vivo transduction efficiency for retinal) for retinal cells (e.g., photoreceptor cells and/or retinal pigment epithelial cells) over existing serotypes;
(6) The recombinant viral particles produced by the variant can have higher transduction activity for retinal cells via intravitreal administration and/or subretinal administration.

Thus, in one embodiment, the present invention relates to an AAV capsid protein variant comprising an engineered capsid protein VP1, said VP1 comprising the following amino acid mutations relative to a parental AAV capsid protein VP1: I240T and V708I.

In some embodiments, the capsid protein variant further comprises a Y444F substitution. In some embodiments, the capsid protein variant further comprises a T491V substitution.

In some embodiments, the capsid protein variant further comprises an insertion of an insert (5mer-15mer) of 5-15 amino acids in AAV variable region VIII (e.g., between positions 587 and 588), preferably no more than 10 amino acids, preferably the insert is 5, 6, 7, 8, 9, or 10 amino acids in length. In some embodiments, the insert may be a new targeting short peptide, or a polypeptide that changes the steric structure for the original receptor binding, or a polypeptide that specifically binds to a potential new receptor, or a polypeptide that attenuates the binding activity of AAV2 to HSPGs. In some embodiments, the insert comprises LALGETTRPA, LALGDVTRPA, or LALGEVTRPA. In some embodiments, the insert may consist of LALGETTRPA, LALGDVTRPA, or LALGEVTRPA. In some embodiments, the inserted amino acids may be a polypeptide with the addition of 1-5 additional amino acids at one or both termini of the amino acid sequence LALGETTRPA, LALGDVTRPA, or LALGEVTRPA, respectively. In some embodiments, the inserted amino acids may be a polypeptide with a deletion of 1-3 amino acids at one or both termini of the amino acid sequence LALGETTRPA, LALGDVTRPA, or LALGEVTRPA, respectively. In some embodiments, the inserted amino acids can consist of a polypeptide having 1-2 amino acid mutations (e.g., substitution mutations or deletion mutations) relative to amino acid short peptide LALGETTRPA, LALGDVTRPA, or LALGEVTRPA, wherein the mutations do not alter or substantially not alter the stability or tissue tropism of AAV viral particles comprising the capsid protein variants of the present invention.

Thus, in some embodiments, the present invention relates to an AAV capsid protein variant comprising an engineered AAV capsid protein VP1 comprising, or consisting only of, the following amino acid mutations relative to a parental AAV capsid protein VP1;
(1) I240T-V708I:
(2) I240T-V708I and an insertion of a fragment LALGETTRPA, LALGDVTRPA, or LALGEVTRPA between positions 587-588;
(3) I240T-V708I-Y 444F;
(4) I240T-V708I-Y444F and an insertion of a fragment LALGETTRPA, LALGDVTRPA, or LALGEVTRPA between positions 587-588;
(5) I240T-V708I-T491V; or
(6) I240T-V708I-T491V and insertion of a fragment LALGETTRPA, LALGDVTRPA, or LALGEVTRPA between positions 587-588.

Parental capsid proteins such as parental capsid protein VP1 may be from a particular AAV serotype (AAV serotype 2 to AAV serotype 12) or a modified version of any of these serotypes, including AAV 4YF and AAV2.7m8 vectors, such as AAV serotype 1 (AAV1), AAV serotype 2 (AAV2), AAV2 modified version 7m8 (AAV2.7m8), AAV serotype 3a (AAV3a), AAV serotype 3b (AAV3b), AAV4 (AAV4), AAV serotype 5 (AAV5), AAV serotype 6 (AAV6), AAV serotype 7 (AAV7), AAV serotype 8 (AAV8), AAV serotype 9 (AAV 9), or AAV serotype 10 (AAV10).

In an exemplary embodiment, the parental capsid protein is a capsid protein of AAV2 or AAV2.7m8 serotype. In an exemplary embodiment, the parent capsid protein VP1 is a capsid protein VP1 of AAV2 or AAV2.7m8 serotype.

In an exemplary embodiment, the capsid protein VP1 of the parental AAV2 serotype comprises or consists of the amino acid sequence as set forth in SEQ ID NO: 1. In an exemplary embodiment, the capsid protein VP1 of the parental AAV2.7m8 serotype comprises or consists of the amino acid sequence as set forth in SEQ ID NO: 6.

In some embodiments, the AAV capsid protein VP1 of the present invention comprises I240T-V708I, and optionally Y444F and/or T491V, as compared to the parental AAV capsid protein VP1, wherein the amino acid positions are determined with reference to the amino acid sequence of AAV2 capsid protein VP1 (SEQ ID NO: 1). In some embodiments, the AAV capsid protein variant of the present invention further comprises an insertion of a fragment LALGETTRPA, LALGDVTRPA, or LALGEVTRPA between positions 587 and 588, as compared to the parent AAV capsid protein, wherein the amino acid positions are determined with reference to the amino acid sequence of AAV2 capsid protein VP1 (SEQ ID NO: 1).

In some embodiments, as compared to a parent AAV capsid protein, the capsid protein variant of the present invention comprises;
I240T-V708I, and an insertion of a fragment LALGETTRPA, LALGDVTRPA, or LALGEVTRPA between positions 587 and 588;
optionally, the capsid protein variant further comprises Y444F and/or T491V;
wherein said amino acid positions are determined with reference to the amino acid sequence of AAV2 capsid protein VP1 (SEQ ID NO: 1).

In some embodiments, the AAV capsid protein variant of the present invention
(i) comprises the amino acid sequence as set forth in SEQ ID NO:2: or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:2, and comprises the substitutions I240T-V708I and the insert 587-LALGETTRPA-588;
(ii) comprises the amino acid sequence as set forth in SEQ ID NO:3: or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:3, and comprises the substitutions I240T-V708I-Y444F and the insert 587-LALGETTRPA-588;
(iii) comprises the amino acid sequence as set forth in SEQ ID NO:4; or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:4, and comprises the substitutions I240T-V708I and the insert 587-LALGEVTRPA-588;
(iv) comprises the amino acid sequence as set forth in SEQ ID NO:5; or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:5, and comprises the substitutions I240T-V708I and the insert 587-LALGDVTRPA-588.

In some embodiments, the AAV capsid protein variant of the present invention is
(i) encoded by the nucleic acid sequence as set forth in SEQ ID NO:7: or encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:7 and comprises the substitution I240T-V708I and the insert 587-LALGETTRPA-588;
(ii) encoded by the nucleic acid sequence as set forth in SEQ ID NO:8: or encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:8 and comprises the substitution I240T-V708I-Y444F and the insert 587-LALGETTRPA-588;
(iii) encoded by the nucleic acid sequence as set forth in SEQ ID NO:9; or encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:9 and comprises the substitution I240T-V708I and the insert 587-LALGEVTRPA-588;
(iv) encoded by the nucleic acid sequence as set forth in SEQ ID NO: 10; or encoded by a nucleic acid sequence having at least 85%, 86%, 87%, 88%, 89%, 90%, 91%, 92%, 93%, 94%, 95%, 96%, 97%, 98% or 99% identity to the nucleic acid sequence as set forth in SEQ ID NO:10 and comprises the substitution I240T-V708I and the insert 587-LALGDVTRPA-588.

In some embodiments, the present invention also relates to a coding nucleic acid of capsid protein variant, comprising a nucleotide sequence encoding the AAV capsid protein variant described herein.

In some embodiments, the coding nucleic acid
(i) comprises or consists of the nucleic acid sequence as set forth in SEQ ID NO:7: or comprises a nucleic acid sequence having at least 85%, 86%, 87%, 88%, 89%, 90%, 91%, 92%, 93%, 94%, 95%, 96%, 97%, 98% or 99% identity to the nucleic acid sequence as set forth in SEQ ID NO: 7, and the capsid protein variant encoded by said nucleic acid sequence comprises the substitution I240T-V708I and the insert 587-LALGETTRPA-588;
(ii) comprises or consists of the nucleic acid sequence as set forth in SEQ ID NO:8: or comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO: 8, and the capsid protein variant encoded by said nucleic acid sequence comprises the substitution I240T-V708I-Y444F and the insert 587-LALGETTRPA-588;
(iii) comprises or consists of the nucleic acid sequence as set forth in SEQ ID NO:9: or comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO: 9, and the capsid protein variant encoded by said nucleic acid sequence comprises the substitution I240T-V708I and the insert 587-LALGEVTRPA-588;
(iv) comprises or consists of the nucleic acid sequence as set forth in SEQ ID NO:10; or comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO: 10, and the capsid protein variant encoded by said nucleic acid sequence comprises the substitution I240T-V708I and the insert 587-LALGDVTRPA-588.

The present invention also relates to a plasmid comprising a nucleic acid encoding a capsid protein variant of the present invention, or comprising a coding nucleic acid of capsid protein variant of the present invention.

### III. Nucleic Acid of Interest

The capsid protein of the present invention can package the nucleic acid of interest to constitute a viral particle.

Nucleic acids of interest suitable for being encoded by the viral particle of the invention are any nucleic acid encoding a therapeutic or prophylactic protein, in particular nucleic acid encoding a protein for the prevention or treatment of ophthalmic diseases, such as an ophthalmic related gene, e.g., RPE65, AIPL1, PROM1, RS1 gene etc. In some embodiments, the protein for preventing or treating ophthalmic diseases include, but are not limited to, e.g., AIPL1, PROM1, RS1, or antibody analogs, and the like.

The nucleic acid of interest may be comprised in an expression cassette and thus packaged within the AAV capsid.

In some embodiments, the expression cassette comprises at least one ITR sequence, thereby allowing the vector genome to be packaged by the AAV capsid. The expression cassette may be single-stranded DNA, double-stranded DNA or single-stranded RNA or double-stranded RNA.

In some embodiments, the expression cassette may comprise one or more regulatory sequences to direct the expression of coding sequence of the nucleic acid of interest in a target cell (e.g., a retinal target cell such as a photoreceptor cell or an optic nerve cell). The regulatory sequence may be selected from the group consisting of a transcription initiation sequence, a termination sequence, a promoter, and/or a enhancer sequence operably linked to a coding sequence: an efficient RNA processing signals such as a splicing and polyadenylation (polyA) region, including a human growth hormone polyadenylation region: an inverted repeat sequence (e.g., L-ITR or R-ITR); a selectable marker or reporter gene, such as a resistance gene; a microRNA: a post-transcriptional regulatory sequence, such as WPRE (a woodchuck hepatitis virus post-transcriptional regulatory element); a cytoplasmic mRNA-stabilized sequence: a nucleic acid restriction site: a homologous recombination sequence: a sequence that enhances translation efficiency (e.g., Kozak consensus sequence); a sequence that enhances protein stability; and a sequence that enhances the secretion of the encoded product when desired.

In some preferred embodiments, the regulatory sequence is located in 5′UTR or 3′UTR. In some preferred embodiments, the regulatory sequence is selected from one or more of the group consisting of:
a promoter, an inverted repeat sequence, an intron, an enhancer, a post-transcriptional regulatory sequence, a polyadenylation region, a selectable marker, or a reporter gene.

Examples of promoters suitable for the present invention include, but are not limited to, the promoters from bacteria, yeast, plants, viruses, and mammals, including simians and humans. The promoter may be constitutive or may be inducible. Constitutive promoters initiate RNA synthesis independent of regulatory influences.

The expression cassettes of the present invention may also comprise a selectable marker or a reporter gene, for example to determine the expression of the vector in a growth system (e.g., bacterial cells) or in target cells. The “selectable marker” or “reporter gene” of the present invention may be selected from those known in the art. Suitable reporter genes include, but are not limited to, an enhanced green fluorescent protein, a red fluorescent protein, luciferase, and secreted embryonic alkaline phosphatase (seAP), which may include sequences encoding geneticin, hygromycin, or puromycin resistance, and the like. Such selectable markers or reporter genes (which may or may not be located outside the viral genome to be packaged into viral particles) can be used to signal the presence of the plasmid in the bacterial cell, e.g., antibiotic resistance marker genes, such as ampicillin or tetracycline resistance or kanamycin resistance.

A “post-transcriptional regulatory sequence” of the present invention is a DNA sequence that, when transcribed, enhances the expression of one or more transgenes or fragments thereof delivered by a viral vector of the present invention. The post-transcriptional regulatory sequence includes, but are not limited to, a hepatitis B virus post-transcriptional regulatory element (HPRE) and a woodchuck hepatitis virus post-transcriptional regulatory element (WPRE). The WPRE is a tripartite cis-acting element that has been shown to enhance transgene expression driven by certain, but not all, promoters.

The expression cassette or expression vector of the present invention may also comprise a polyadenylation region, such as the hGHpA (a human growth hormone polyadenylation region).

The expression cassette or expression vector of the present invention may also comprise an intron, such as a chimeric intron.

### IV. Viral Particle

The present invention thus relates to a recombinant AAV viral particle (rAAV) comprising
(i) AAV capsid protein variants of the present invention; and
(ii) the nucleic acid of interest, e.g., an ophthalmic disease-related gene or a coding nucleic acid for a protein for treating an ophthalmic disease, packaged within the AAV capsid.

### V. Preparation Method

The present invention relates to a method for preparing recombinant AAV viral particles (rAAV). Many methods are known in the art for packaging and producing rAAV. Currently, the commonly used rAAV packaging systems mainly include a triple-plasmid co-transfection system, a system with adenovirus as a helper virus, a packaging system with herpes simplex virus type 1 (HSV1) as a helper virus, and a baculovirus-based packaging system. Each packaging system has its own characteristics, and those skilled in the art can make appropriate selections according to needs.

The rAAV production cultures for production of rAAV viral particles all require: 1) suitable host cells including, for example, human-derived cell lines such as HEK-293T cells, or insect-derived cell lines (in the case of baculovirus production systems): 2) suitable helper virus functions provided by wild-type or mutant adenoviruses (e.g., temperature-sensitive adenoviruses), herpes viruses, baculoviruses or plasmid constructs providing helper functions: 3) AAV genes rep and cap and gene products: 4) genes/nucleic acids of interest flanked by at least one AAV ITR sequence, and preferably driven by an operably linked promoter; and 5) suitable culture systems to support rAAV production.

In some embodiments, the present invention relates to a method of producing a recombinant AAV viral particle, comprising culturing a packaging cell under conditions sufficient for production of the recombinant AAV viral particle, wherein the packaging cell comprises a plasmid comprising a nucleic acid encoding the capsid protein variant of the present invention or the capsid protein variant coding nucleic acid of the present invention.

In some embodiments, the packaging cell further comprises a helper plasmid and/or a transfer plasmid comprising the nucleic acid of interest.

In some embodiments, the method further comprises isolating from the culture supernatant a self-complementary recombinant AAV viral particle.

In some embodiments, the method further comprises lysing the packaging cell, and isolating a recombinant AAV viral particle from the cell lysate.

In some embodiments, the method further includes one or more of the following steps:
a. removing the cell debris,
b. treating the supernatant containing the recombinant AAV viral particle with a benzonase nuclease,
c. concentrating the recombinant AAV viral particle,
d. purifying the recombinant AAV viral particle.

Thus, the present invention also relates to a packaging cell for producing a recombinant AAV viral particle, said packaging cell comprising a plasmid comprising a nucleic acid encoding the capsid protein variant of the present invention or a capsid protein coding nucleic acid of the present invention or the expression cassette of the present invention.

### VI. Composition, Medicament or Formulation

The present invention provides a formulation, composition or medicament, which contains (a) the rAAV of the present invention, and (b) pharmaceutically acceptable auxiliary materials, such as pharmaceutically acceptable carriers and excipients known in the art, including buffers.

As used herein, “pharmaceutically acceptable carrier” includes any and all solvents, dispersion medium, isotonic agent and absorption delaying agents, and the like that are physiologically compatible. See also “Handbook of Pharmaceutical Excipients”, 8th Edition, R. C. Rowe, P. J. Seskey and S. C. Owen, Pharmaceutical Press, London, Chicago for the use of pharmaceutically acceptable auxiliary materials and uses thereof.

In some embodiments, the pharmaceutically acceptable auxiliary materials include, but are not limited to, one or more compatible solid or liquid fillers or gel materials that are suitable for human use and must be of sufficient purity and sufficiently low toxicity. By “compatible” is meant herein that the components of the composition are capable of admixing with and between the active ingredients of the present invention without significantly diminishing the efficacy of the active ingredients. Suitable pharmaceutically acceptable auxiliary materials will be known to those skilled in the art. Examples of pharmaceutically acceptable carrier moieties are cellulose and its derivatives (e.g., sodium carboxymethylcellulose, sodium ethylcellulose, cellulose acetate, etc.), gelatin, talc, solid lubricants (e.g., stearic acid, magnesium stearate), calcium sulfate, vegetable oils (e.g., soybean oil, sesame oil, peanut oil, olive oil, etc.), polyols (e.g., propylene glycol, glycerin, mannitol, sorbitol, etc.), emulsifiers (e.g., Tween®), wetting agents (e.g., sodium lauryl sulfate), colorants, flavors, stabilizers, antioxidants, preservatives, pyrogen-free water, and the like.

The formulation or composition or medicament of the present invention may be a liquid or a solid, such as a powder, gel or paste. Preferably, the formulation or composition or medicament of the present invention is a liquid, preferably an injectable liquid. Preferably, the injectable liquid is provided as a capsule or in a syringe.

The rAAV of the present invention, or a formulation, composition, or medicament comprising the same, may be administered intravenously, intramuscularly, subcutaneously, orally, by mucosal contact, intraperitoneally, and intralesionally, preferably topically to the eye, for example by intraretinal or intravitreal administration, such as intravitreal injection, subretinal injection, or suprachoroidal injection. In some embodiments, the rAAV of the present invention, or a formulation or composition or medicament comprising the same, may be administered intraretinally or intravitreally, e.g., by intravitreal or subretinal administration, e.g., intravitreal administration (IVT administration). In either mode of administration, preferably, the formulation or composition or medicament of the present invention is provided as an injectable liquid.

The composition or formulation or medicament may comprise physiologically acceptable sterile aqueous or anhydrous solutions, dispersions, suspensions or emulsions, and sterile powders for reconstitution into sterile injectable solutions or dispersions. Suitable aqueous and nonaqueous carriers, diluents, solvents or excipients include water, ethanol, polyols and suitable mixtures thereof.

The composition, e.g., pharmaceutical composition or pharmaceutical formulation, of the present invention may further comprise additional active ingredients, e.g., one or more additional therapeutic agents, e.g., an immunomodulator, e.g., an immunosuppressant.

### VII. Combination Product

In one aspect, the present invention also provides a combination product (e.g., a pharmaceutical combination product) comprising the rAAV of the present invention, and one or more additional therapeutic agents. The combination product of the present invention may be used in the therapeutic method of the present invention.

The present invention also provides a pharmaceutical kit comprising the combination product, e.g. the pharmaceutical kit comprises the following in the same package:
a first container containing the rAAV of the present invention or a medicament comprising said rAAV:
a second container containing a pharmaceutical composition comprising one or more additional therapeutic agents, e.g., an immunomodulator.

In some embodiments, the additional therapeutic agent is an immunomodulator, e.g., an immunosuppressive agent, e.g., for reducing an immune response, such as an immunoinflammatory response, generated by the rAAV viral particle.

### VIII. Therapeutic Method

In one embodiment, the rAAV, formulation or composition or medicament of the present invention is used for the treatment of an ocular disease.

In some embodiments, the ocular disease includes, but are not limited to, for example, congenital cataracts, glaucoma, congenital coloboma of the retina, iris, or choroid, retinitis pigmentosa, retinoblastoma, pathological myopia, congenital optic neuropathy, strabismus, keratoconus, and the like.

In one embodiment, the rAAV, formulation or composition or medicament of the present invention is administered intraocularly, e.g., via intraretinal or intravitreal administration, e.g., via subretinal or intravitreal administration In one embodiment, the administration is injection.

In one embodiment, the present invention also relates to the use of a recombinant AAV viral particle, a formulation or composition or a combination product comprising the same, in the manufacture of a medicament for the treatment of an ocular disease according to the present invention.

Any documents cited herein, including patents, patent applications, and literature, are incorporated herein in their entirety.

Any or all of the features discussed above and throughout this application may be combined in various embodiments of the present invention. The following examples further illustrate the present invention, however, it should be understood that the examples have been described by way of illustration and not limitation, and are not intended to, and should not be construed to, limit the scope of the invention in any way, the scope of the present invention, and that various modifications may be made by those skilled in the art.

### EXAMPLE

### Example 1: Construction of Capsid Plasmids for Four Serotypes RC-C02, RC-C03, RC-C13, and RC-C14, and Virus Packaging and Detection of Virus Yield

### Construction of RC-C02 Serotype Capsid Plasmid

The VP1 gene and the downstream part poly sequence of the AAV2 serotype plasmid pRC2 (synthesized by Genescript, comprising the pRC plasmid of AAV2 serotype VP1 (SEQ ID NO:1)) were completely digested with SwaI at 2033 bp and SmaI at 4348 bp to obtain a linearized vector, the VP1 gene and downstream part poly sequence of AAV2 were removed and replaced with a 2375 bp SwaI/SmaI fragment containing the RC-C02 VP1 and the downstream part poly sequence by homologous recombination, wherein the fragment was amplified by polymerase chain reaction (PCR) with pRC2 plasmid as a template, 4 amplified products were obtained as follows by PCR: (a) The upstream fragment of the I240T mutation region, with 5′ terminus amplification primer as RC-C02-F1, AACAATAAATGATTTAAATCAGGTATGG (SEQ ID NO:11), and 3′ terminus amplification primer as RC-C02-R1, GTGGTGGTGACTCTGTCGCCCATCCATG (SEQ ID NO: 12). (b) The downstream of the I240T mutation region and the upstream of the mutation region where the position 588aa was inserted with 10aa, with 5′ terminus amplification primer as RC-C02-F2, ACAGAGTCACCACCACCAGCACCCGAACC (SEQ ID NO:13), and 3′ terminus amplification primer as RC-C02-R2, TTGTTGTTTCGCCGAGTGCTAGGTTGCCTCTCTGGAGGTTG (SEQ ID NO:14). (c) The downstream of the mutation region where the position 588aa was inserted with 10aa and the upstream of the V708I mutation region, with 5′ terminus amplification primer as RC-C02-F3, TCGGCGAAACAACAAGACCTGCTAGGCAAGCAGCTACCGCAG (SEQ ID NO:15), and 3′ terminus amplification primer as RC-C02-R3, ACATTAATAGACTTGTTGTAGTTGGAAG (SEQ ID NO: 16). (d) The downstream of the V708I mutation region, with 5′ terminus amplification primer as RC-C02-F4, ACAAGTCTATTAATGTGGACTTTACTGTGG (SEQ ID NO:17), and 3′ terminus amplification primer as RC-C02-R4, GCTGTTTAAACGCCCGGGCTGTAG (SEQ ID NO: 18); and the above 4 amplification products were overlapped to obtain a 2375 bp SwaI/SmaI fragment containing RC-C02 VP1 and part of poly sequence downstream.

### Construction of RC-C03 Serotype Capsid Plasmid

Based on the cloned pRC-C02 plasmid, the VP1 gene and part of poly sequence downstream of the RC-C02 plasmid were digested with SwaI at 2033 bp and SmaI at 4378 bp to obtain a linearized vector, the VP1 gene and the downstream part poly sequence of AAV2 were removed and replaced with a 2375 bp SwaI/SmaI fragment containing the RC-C03 VP1 and the downstream part poly sequence by homologous recombination, wherein the fragment was obtained by obtaining 2 fragment products as follows by amplifying the RC-C02 plasmid by PCR: (a) The upstream of the Y444F mutation region, with 5′ terminus amplification primer as RC-C02-F1, AACAATAAATGATTTAAATCAGGTATGG (SEQ ID NO:11), and 3′ terminus amplification primer as RC-C03-R1, GCTCAAGAAATACAGGTACTGGTCGATG (SEQ ID NO: 19). (b) The downstream of the Y444F mutation region, with 5′ terminus amplification primer as RC-C03-F2, CCTGTATTTCTTGAGCAGAACAAACACTC (SEQ ID NO: 20), and 3′ terminus amplification primer as RC-C02-R4, GCTGTTTAAACGCCCGGGCTGTAG (SEQ ID NO: 18); and the 2 amplification products were overlapping amplified to obtain a 2375 bp SwaI/SmaI fragment containing RC-C03 VP1 and the downstream part poly sequence.

### Construction of RC-C13 Serotype Capsid Plasmid

With the cloned pRC-C02 plasmid as a template, the AAV2 VP1 gene and the downstream part poly sequence were completely digested with SwaI at 2033 bp and SmaI at 4378 bp to obtain a linearized vector, the cap gene and the downstream part poly sequence of AAV2 were removed and replaced with a 2375 bp Swa I/Sma I fragment containing RC-C13 cap and the downstream part poly sequence by homologous recombination, wherein the fragment was obtained by obtaining 2 fragment products as follows by amplifying the RC-C02 plasmid by PCR: (a) The upstream of the T593V mutation region, with 5′ terminus amplification primer as RC-C02-F1, AACAATAAATGATTTAAATCAGGTATGG (SEQ ID NO:11), and 3′ terminus amplification primer as RC-C13-R1, GTCTTGTTACTTCGCCGAGTGCTAGGTTG (SEQ ID NO:21). (b) The downstream of the T593V mutation region, with 5′ terminus amplification primer as RC-C13-F2, CACTCGGCGAAGTAACAAGACCTGCTAGGCAAG (SEQ ID NO:22), and 3′ terminus amplification primer as RC-C02-R4, GCTGTTTAAACGCCCGGGCTGTAG (SEQ ID NO:18); and the above 2 amplification products were overlapped to obtain a 2375 bp SwaI/SmaI fragment containing RC-C13 cap and the downstream part poly sequence.

### Construction of RC-C14 Serotype Capsid Plasmid

Based on the RC-C02 serotype capsid plasmid obtained by the above cloning, the VP1 gene and the downstream part poly sequence of pRC-C02 plasmid were completely digested with SwaI at 2033 bp and SmaI at 4378 bp to obtain a linearized vector, the cap gene and the downstream part poly sequence of AAV2 were removed and replaced with a 2375 bp Swa I/Sma I fragment containing RC-C14 VP1 and the downstream part polyA sequence by homologous recombination, wherein the fragment was obtained by obtaining 2 fragment products as follows by amplifying the RC-C02 plasmid by PCR: (a) the upstream of the E592D. T593V mutation region, with 5′ terminus amplification primer as RC-C02-F1, AACAATAAATGATTTAAATCAGGTATGG (SEQ ID NO: 11), 3′ and terminus primer amplification as RC-C14-R1. TCTTGTTACATCGCCGAGTGCTAGGTTGC (SEQ ID NO:23). (b) E592D, the downstream of the T593V mutation region, with 5′ terminus amplification primer as RC-C14-F2, TCGGCGATGTAACAAGACCTGCTAGGCAAG (SEQ ID NO:24), and 3′ terminus amplification primer as RC-C02-R4, GCTGTTTAAACGCCCGGGCTGTAG (SEQ ID NO:18); the above 2 amplification products were overlapped to obtain a 2375 bp SwaI/SmaI fragment containing pRC-C14 VP1 and the downstream part poly sequence.

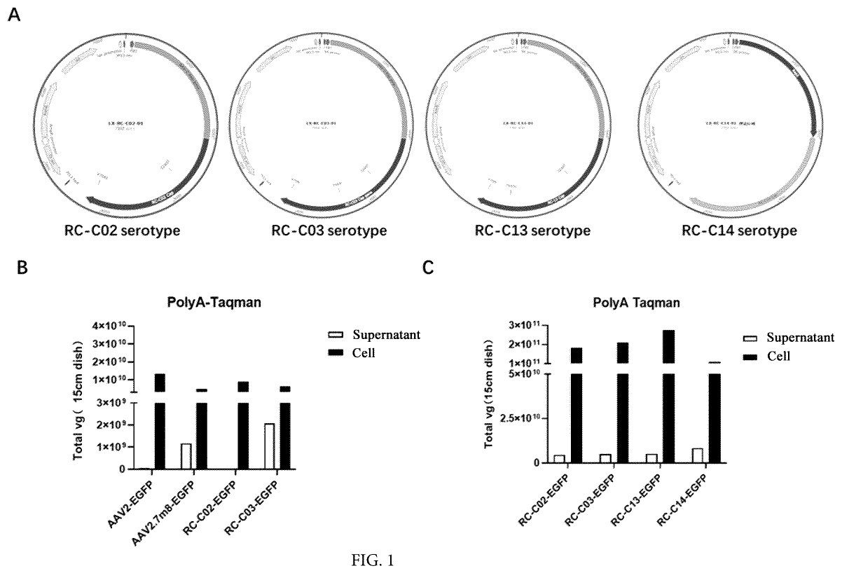

The results of Sanger sequencing show that RC-C02/RC-C03/RC-C13/RC-C14 serotype VP1 gene sequences were successfully constructed on pRC2 vectors to form pRC-C02, pRC-C03, pRC-C13, and pRC-C14 (a plasmid map drawn by Snapgene as shown in a FIG. 1A), respectively, and the newly constructed four serotype plasmids were further subjected to virus packaging.

### Detection Method for AAV Virus Titer:

Preparing a standard sample: a single restriction site of a GOI-E04 (synthesized by Genescript (GOI-E04 containing CAG-EGFP expression cassette, with sequence thereof as set forth in SEQ ID NO: 25)) was selected, followed by cutting gel after restriction enzyme digestion to recover linear DNA, DNA concentration of the recovered product was measured by using Nanodrop, copy number was calculated according to the formula c (copy/ul)=plasmid concentration (ng/ul)*(1e-9)*Avogadro constant/(660 g/mol*plasmid base-pair number), the plasmid was diluted to 1e9 copy/ul, and cryopreserved at −80° C. after subpackaging.

Diluting a standard sample: a sub-packaged standard sample was taken and diluted to 1e8, 1e7, 1e6, 1e5, 1e4, 1e4 and 1e2 copy/ul by ddH2O to be used as a standard sample template. Preparation and dilution of sample: the mixture was prepared according to the following system, Benzonase 2.5U, MgCl2 with final concentration 2 mM, virus Sul, supplementing with ddH2O to 49 ul, the mixture was gently blended, centrifuged, treated at 37° C. for 1 h, and then treated at 85° C. for 20 min. 1 ul of proteinase K (10 mg/ml) was added, the mixture was gently blended by shaking, centrifuged, treated at 55° C. for 10 min, and then treated at 85° C. for 20 min. The treated samples (10-fold diluted samples) were further diluted 100-fold and 500-fold to obtain 1000- and 5000-fold diluted samples, with the 1000- and 5000-fold diluted samples as templates to be tested.

The four serotype plasmids prepared as described above were co-transfected with GOI-E04 & Helper plasmids into HEK293T cells for 72 h, the cells and the supernatant were collected for real-time fluorescence quantitative PCR (qPCR) using polyA as a probe to detect the copy number of rAAV nucleic acid in the supernatant and cells. The specific process is as follows:

Firstly, HEK293T cells were allowed to adhere to the culture dish after recovery, and when the confluency of HEK293T cells in the culture dish reached 90%, the cell line was passaged at a ratio of 1:3, followed by culturing for 24 h until the cell confluence reaches 80%, and carrying out plasmid transfection: suitable transfection systems were prepared before transfection, and for each 10 cm dish of the packaging system, the transfection mixture was preferably prepared according to the following system: serum-free Medium Opti-MEM (Gibco) 500 ul, Helper plasmid 15 μg (synthesized by Genescript), plasmids of four serotype prepared as described above 7.5 μg, GOI plasmid 7.5 μg, PEIpro (Polyplus) 22.5 ul; thirdly, transfection process: the transfection mixture was dropwise added into different areas of a 10 cm culture dish, followed by shaking up in a gentle cross manner; and fourthly, packaging and culturing: the transfected cells were transferred to a carbon dioxide incubator, and cultured for 72 h at 37° C.; and finally, collecting virus: 72 h after transfection, the cells were pipetted with cell supernatant and centrifuged at 1500 rpm to collect cell pellets; lysate was added for lysis in a shaker at 37° C. for 1 h, followed by centrifuging at 4000 rpm with a horizontal rotor for 10 min, the supernatant was collected, filtered with 0.45 μm needle filter, and subpackaged in 10 ul volume, and virus gene copy number (unit: vg) in cells was detected by real-time quantitative PCR method.

Methods of QPCR Reaction and Titer Calculation were as Follows:

Preparation system (20 ul): 2× Probe Premix 10 ul, 10 uM hGHpA primers F and R 0.4 ul each, hGHpA Probe 0.8 ul. 50×ROX II 0.4 ul, template 2 ul, and supplementing water to 20 ul. The detection was performed according to the program: 95° C. for 5 min, 95° C. for 5 s, 60° C. for 30 s, for 40 cycles; after the data was output, the titer (VG/ml)=output result*dilution multiple*1000.

As shown in FIG. 1: in the adherent 293T, AAV2 and AAV2.7m8 (RC-C01) viruses were used as experimental controls, and the adherent 293T was used for virus production testing. From the results of FIGS. 1B and 1C, it can be found that the virus formation of the four new serotypes RC-C02, RC-C03, RC-C13, and RC-C14 is mainly in the cytoplasm, with limited amount of virus externally secreted in the supernatant. At the same time, compared with the two control viruses, the crude virus yield of the four mutants did not decrease significantly.

As shown in the rational design scheme for capsid VP1 as described herein, the amino acid number of RC-C02 capsid (Cap) is 735, the amino acid number of RC-C03 serotype Cap is 735 as well, the amino acid number of RC-C13 serotype Cap is 745, and the amino acid number of RC-C14 serotype Cap is 745. According to the capsid sequence of pRC2 (AAV2), amino acid sequences of about 100 serotype capsid were generated through computer simulation design, analysis, and directional design. After virus preparation and production, they were screened and verified in cell lines (293T, CHO, ARPE19, 661W, etc.) in vitro, and the above-mentioned 4 new serotypes were obtained. Compared with the known AAV2, which is widely used in ophthalmic gene therapy, and AAV2.7m8 with the best retinal transduction activity, the two serotypes of RC-C14 and RC-C02 showed stronger tropism for retinal tissue.

### Example 2: Comparison of Transduction Activity of AAV2 and Variants Thereof in Different Cell Lines

The steps for flow cytometry detection of AAV virus TU are as follows:

1. Adherently cultured HEK293T (or ARPE19/661W/CHO) cells to be infected, and purified viruses of the four serotypes AAV2, AAV2.7m8, RC-C02, and RC-C03 were prepared.

2. Day 1 Cell plating; after digestion and detachment, cells were collected by centrifugation at 1000 rpm for 5 min, followed by resuspension for counting, and plating in 96-well plates as TIE+4/well.

3. Day 2 Viral infection: the cells were counted after being plated for 24 hours;

AAV was diluted by complete medium (diluent) in a 10-fold serial dilution, according to the following dilution ratio:

$$1 \times 1 {0}^{- 2} = 990 μL of diluent + 10 μL of stock solution$$

$$1 \times 1 {0}^{- 3} = 900 μL of diluent + 100 μL of the previous diluent$$

$$1 \times 1 {0}^{- 4} = 900 μL of diluent + 100 μL of the previous diluent$$

$$1 \times 1 {0}^{- 5} = 900 μL of diluent + 100 μL of the previous diluent$$

$$1 \times 1 {0}^{- 6} = 900 μL of diluent + 100 μL of the previous diluent$$

$$1 \times 1 {0}^{- 7} = 900 μL of diluent + 100 μL of the previous diluent$$

Cells cultured overnight were taken, after pipetting the complete medium from the wells, 100 μL of medium containing serially diluted viruses was added (8 wells per dilution), the cells mixture was cultured in a 37° C., 5% CO₂ cell incubator for 72 hours.

5. Day 5 Taking photos of digested cells; after 72 hours, the medium was discarded, 100 μL of PBS was added to wash the cells, and the PBS was discarded, 20 μL of Typsin was added, the cells was digested in an incubator at 37° C. for 3 min, the digestion was stopped by adding 80 μL of DMEM containing 10% FBS, followed by flow cytometry detection of virus fluorescence.

6. Data were exported, the flow cytometry results were analyzed and processed by FlowJo for generating an excel file, and a histogram was plotted using Graphpad.

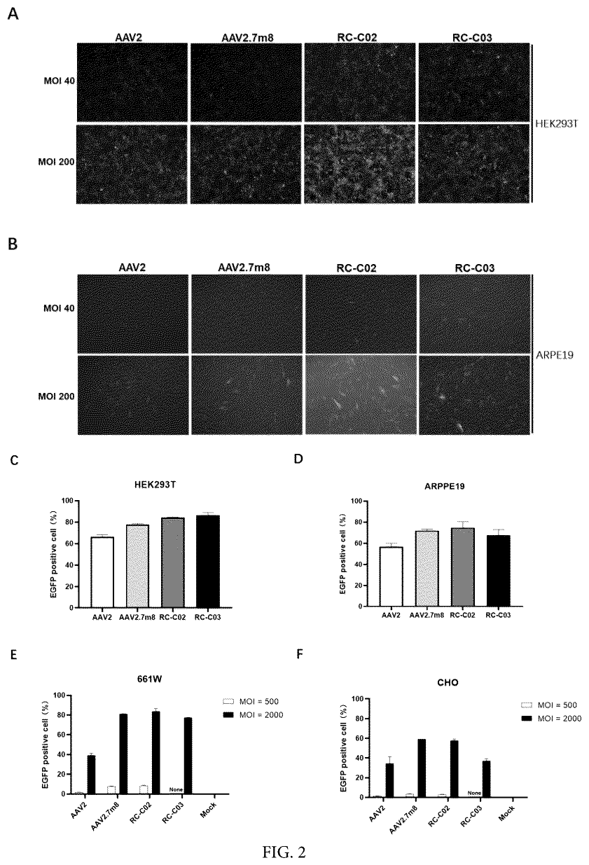

FIG. 2 shows a comparison of the transduction activity of AAV2 and variants thereof in HEK293T, ARPE19, 661W, and CHO cells at different MOIs. From the results of the fluorescence photographs (Nikon fluorescence microscope) of FIGS. 2A-2B, it was found that transduction efficiency of RC-C02 was the best in ARPE19 cells, and that the transduction efficiency of RC-C02 and RC-C03 serotypes in 293T was superior to that of the control group. FIGS. 2C-2D show the statistics results of flow cytometry (FACS) detection, indicating that the percentage of total green fluorescent cells was consistent with that of the fluorescence photographs, and that the transduction activity of the RC-C02 in cell line in vitro was significantly stronger than that of the AAV2 control group. In ARPE19, 661W, CHO (2D, 2E, 2F) cells: the transduction efficiency of RC-C02 and AAV2.7m8 serotype was significantly higher than that of AAV2 and RC-C03, showing the stronger transduction activity for the photoreceptor cells. In 293T (FIG. 2C), the transduction efficiency of RC-C03 was the strongest, and the transduction efficiency of RC-C02 and AAV2.7m8 were both significantly better than that of AAV2 serotype. The statistical results showed that the GFP fluorescence percentage of RC-C02 serotype in 661w was significantly higher than that of the control group virus.

### Example 3: Comparison of In Vitro Transduction Activity of RC-C14 and Variants Thereof

The process for flow cytometry assay for AAV virus transduction activity in CHO, ARPE19, 661W and HEK293T cells is as follows:

1. Preparation of cells to be infected; four lines, HEK293T (available from ATCC), CHO (available from ATCC), ARPE19 (available from ATCC), and 661w (available from Lonza) are all mycoplasma-free. The four viruses carrying fluorescent reporter genes, RC-C02&GOI-E04, RC-C03&GOI-E04, RC-C13&GOI-E04, and RC-C14&GOI-E04 (referred to as RC-C02, RC-C03, RC-C13, RC-C14 in figures), purified by ultracentrifugation, were tested for gene copy number (qPCR method) and stored at −80° C. after recording.

2. Day 1: Cell plating: the cells to be detected were digested, centrifuged at 1000 rpm for 5 min, counted, and plated in a 96-well plate, with HEK293T 1E+4/well.

3. Day 2: Viral infection: the cells were counted after plated for 24 h and AAV was diluted by complete medium (diluent) in serial dilution. The total number of cells in the 96-well plate before infection was used as the base number, and two MOI gradients were set according to the infectivity of each cell type.

Cells cultured overnight were taken, after pipetting the complete medium, 100 μL of diluted virus medium was added (duplicate wells per gradient): the cells mixture was cultured in a 37° C., 5% CO2 cell incubator at for 72 hours.

4. Day 5: Taking photos of digested cells: the medium was discarded, 100 μL of PBS was added to the cells, and the PBS was discarded, 20 μL of Typsin was added, the cells was digested in an incubator at 37° C. for 3 min, the dissociation was stopped by adding 80 μL of DMEM containing 10% FBS, followed by flow cytometry detection of virus fluorescence.

5. Data were exported, the flow cytometry results were analyzed and processed by FlowJo for generating an excel, and a histogram was plotted using Graphpad.

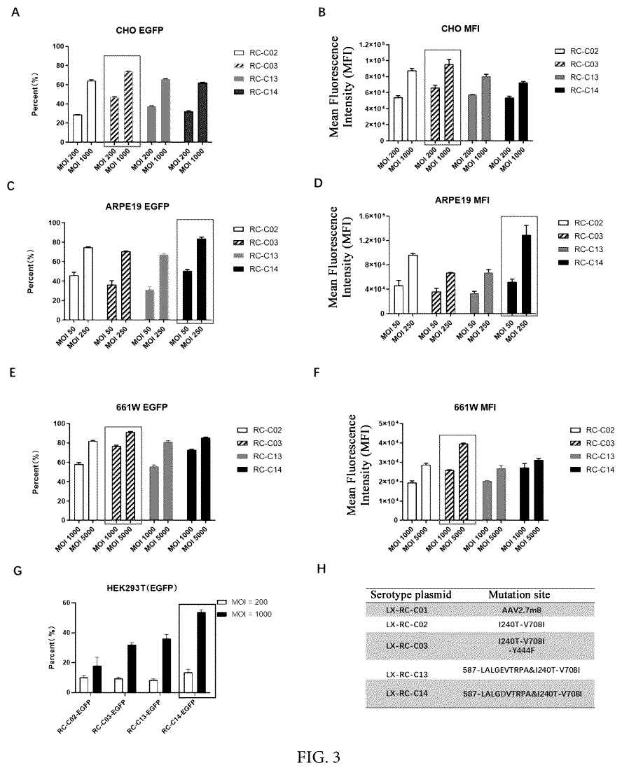

The results in FIG. 3 are statistical results of flow cytometry, showing in vitro transduction activity assays of the four variants performed in CHO, ARPE19, 661W and HEK293T, respectively. The harvested cells were subjected to FACS assay (Beckman, FITC excitation channel) separately after different cell lines were infected with rAAV at two multiplicity of infections for 72 hours.

FIG. 3A represents the percentage of green fluorescent cells in CHO cells, and FIG. 3B represents the average fluorescence intensity in CHO cells. From the statistical results, it can be found that the RC-C03 has the strongest transduction activity in CHO cells (intensity of transduction activity: C03>C13>C14>C02), while in retinal pigment epithelial cells (ARPE19): RC-C14 serotype has the strongest transduction activity (including the percentage of fluorescent cells and the average fluorescence intensity), followed by RC-C02, which is stronger than that of RC-C03 and RC-C13.

FIGS. 3E-3G show an assessment of the transduction activity of the four serotypes disclosed in the present invention in 661W and 293T cells, with RC-C14 serotype having the best cell tropism to HEK293T at multiplicity of infection (MOI) of 1000 (FIG. 3G), and in vitro transduction activity ranked as: RC-C14>RC-C13>RC-C03>RC-C02.

Whereas in 661W cells (3E-3F), the RC-C03 serotype has the highest transduction percentage and mean fluorescence intensity at an MOI of 1000, but showing weaker transduction activity in 293T, indicating that the RC-C03 serotype possessed the tropism for photoreceptor cells. FIG. 3H shows detailed description information of the mutation sites of the VP1 genes of the four serotypes RC-C02, RC-C03, RC-C13, and RC-C14. As described in Example 1, the RC-C02 serotype is a mutation of I to T at position 240 and V to I at position 708 of VP1 of AAV2.7m8: the RC-C03 serotype is a mutation of Y to F at amino acid position 444 of VP1 of RC-C02; the RC-C13 serotype has a insertion of LALGEVTRPA 10-mer peptide after amino acid position 587, and a mutation of I to T at position 240 and V to I at position 708 of VP1 of AAV2; and the RC-C14 serotype is a mutation of E to D at amino acid position 562 of VP1 of RC-C13.

### Example 4: Comparison of In Vivo Transduction Activity of RC-C02 and RC-C14 in Retinal Tissue with Existing Serotypes

### In Vivo Autofluorescence (AF) Detection Method:

Experimental procedure for in vivo autofluorescence detection (AF) is as follows: firstly, the 6-8-week-old C57 mice (supplied by GemPharmatech Co., Ltd.) were examined (three mice per group, 3 groups, both eyes were dosed: 6 eyes in each group were examined per group for AF). After the ear tag was checked, mydriatic drug was dripped on the ocular surface of both eyes, the mice were anesthetized by using Zoletil® mixture according to the anesthetic dose of 60 mg/kg, surface anesthetic was dripped on the ocular surface of both eyes, and the ocular surface was coated with gel to wear a corneal contact lens. The HRA control panel of the inspection equipment was switched into an IR mode, the mouse fundus was focused until the image was clear, then the mode was switched to an FA mode, the SENS value was adjusted to 100, the focal distance was adjusted to be capable of clearly seeing the blood vessels of the retina, the SENS value was lowered to 60, followed by taking photos to ensure that the exposure intensity of the photos is within a reasonable interval.

The AAV2.7m8 serotype recorded in existing literature has a significantly higher transduction efficiency in mouse retinal tissue than that of AAV2 under IVT intraocular administration, so it can be used as a comparison object for the technical product of the present invention. As a further engineered version of AAV2.7m8, the RC-C02 serotype described in the invention has a further improved transduction activity in photoreceptor cells (Example 2). Similarly, AAV2.GL has been reported in existing literature and was obtained through multiple rounds of screening under pressure of peptide display libraries in animals. After IVT injection into the eye, AAV2.GL also has strong retinal tissue transduction activity. AAV2.GL was also obtained by engineering AAV2-VP1, with the specific mutation of an insertion of a 12-mer peptide (AAAGLSPPTRAA) after the amino acid at position 587 of the AAV2-VP1 protein.

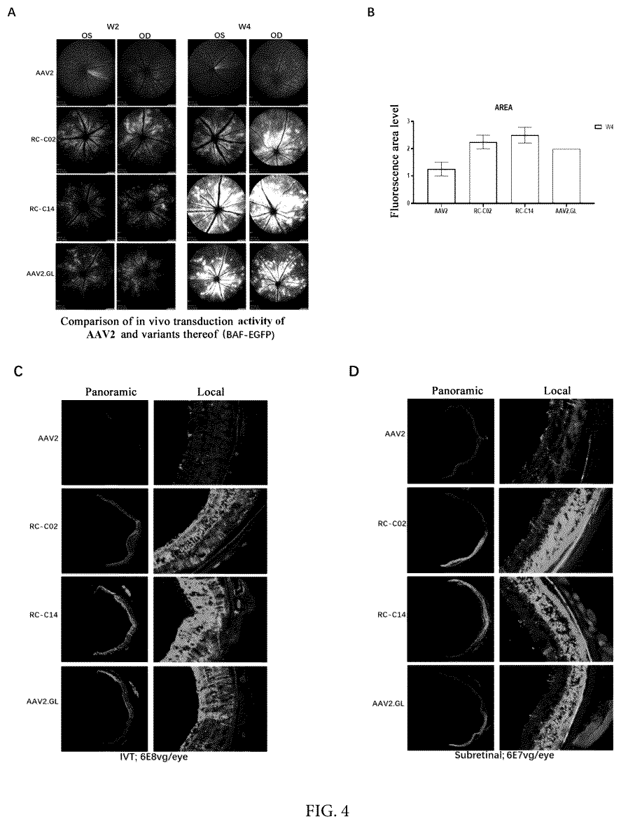

FIG. 4 shows a comparison of tissue distribution and transduction activity of two serotypes RC-C02 and RC-C14 with those of vectors of the existing serotype in the intraocular retinal tissue via IVT intravitreal injection, the four serotypes as described above (AAV2, RC-C02, RC-C14, AAV2.GL) were administered intravitreally, respectively. The transduction activity of AAV virus in the retinal tissue was evaluated 4 weeks after administration, with the virus dose per eye of 6E8vg. At the same time, the intensity of autofluorescence of the left and right fundus of each group of mice was detected in vivo at 2 and 4 weeks after administration. It can be observed from FIG. 4A that two weeks after IVT administration, the spontaneous green fluorescence signals of the fundus of mice corresponding to all of the four serotypes could be detected, wherein the fluorescence signal of the AAV2 administration group was the weakest, and the signal of the RC-C02 administration group was slightly stronger than that of other serotypes. The BAF detection results 4 weeks after intravitreal administration in FIG. 4A showed that, except for the AAV2 administration group, the fundus fluorescence signals of mice in the administration groups of other three serotypes (RC-C02, RC-C14, and AAV2.GL) were significantly enhanced compared with that after 2 weeks, with the fundus fluorescence intensity and fluorescence area of mice in the RC-C14 administration group being significantly higher than those of the other three serotypes (FIG. 4A).

The eyeballs of mice administered for 4 weeks were further sampled and frozen sections of in vitro tissues were made. RPE65 antibody (available from Abcam) was used for labeling and staining (the RPE cell layer labeled in red, the nucleus stained with DAPI, and green fluorescence representing virus-infected cells). From the immunofluorescence images of mouse retinal tissues, it can be found that, with the IVT administration mode (FIG. 4C), except for the control group (AAV2), all of the other three serotypes (RC-C02, RC-C14, and AAV2.GL) were able to fully infect the inner layer of the retina tissues (ganglion cell layer, nerve fiber layer, inner nuclear layer). It can be clearly observed from the panoramic images of retinal stitched by the LifeEvos device that the green fluorescence signal in the RC-C14 administration group is most widely distributed in the inner layer of the retina, and the fluorescence brightness is the strongest. Further comparison and analysis of the fluorescence of the outer layer of the retina, it can be found that, only RC-C14 can fully transduce the retinal outer plexiform layer, outer nuclear layer, and part of the inner segments of the photoreceptor layer (PR). However, under the SR administration mode (FIG. 4D), except for the control group AAV2, the other three serotypes can fully infect the outer layer of the retina (RPE, PR, outer nuclear layer, outer plexiform layer). From the stitched large picture, it can be found that the RC-C02 and RC-C14 administration groups are mainly widely distributed in the outer layer of the retina, and the green fluorescence brightness is stronger than that of the control group AAV2. Further comparison and analysis of the infection of each layer of the retina show that RC-C02 can fully transduce the inner plexiform layer, inner nuclear layer, and part of the ganglion cells (GCL).

### Example 5: Comparison of Tissue Distribution and Differences in Transduction Efficiency of RC-C14 and Variants Thereof (RC-C30&RC-C31) after Intraocular Administration

The RC-C30 serotype described in FIG. 5 is a serotype in which the amino acid at position 444 of the RC-C14 capsid protein VP1 is mutated from Y to F and the amino acid at position 491 is mutated from T to V, while RC-C31 is a serotype in which the 10-mer peptide amino acids (LALGDVTRPA) inserted at position 587 of the RC-C14 capsid protein VP1 are inserted between 579V-590S of VRIII.

When RC-C14 and variants thereof RC-C30 and RC-C31 were administered by IVT (E9vg/eye) and subretinal (E8vg/eye), respectively (as described in Example 4), it was found that AAV2 and RC-C31 were almost rarely infected in AF fundus fluorograms 2 weeks and 4 weeks after IVT administration. The fluorescence distribution and intensity of RC-C14 2 weeks after administration were close to those of mutant RC-C30. After 4 weeks of administration, the fundus examination results in the RC-C14 administration group showed a trend of significantly increased fluorescence intensity, while the intensity of BAF fundus green fluorescence in the RC-C30 group did not show significant improvement. These results of comparation indicate that mutations in the tyrosine and threonine sites on the surface of the AAV capsid do not significantly improve the transduction efficiency of the virus in the ocular tissue, especially via intravitreal administration mode. At the same time, the transduction activity of RC-C30 shows a weakening trend as the extension of administration time (4 weeks after administration), indirectly indicating that there may be proteases that recognize and degrade phenylalanine F and valine V in the components of the vitreous humor in the mouse eye. This further indicates that the amino acid Y at position 444 and the amino acid T at position 491 of the VP1 protein of the RC-C14 serotype are essential for the maintenance of stability of the viral capsid in the tissue fluid. At the same time, the amino acids Y and T exposed on the capsid surface are necessary for AAV to quickly uncapsidate after entering the cell and are essential for the genetic material to enter the nucleus. The presence of these specific amino acids on the capsid surface improves the transduction efficiency of AAV virus in retinal tissue, avoids degradation by proteases in the tissue fluid, and improves the timeliness of AAV genetic material expressing exogenous genes.

### Example 6: Comparison of the Distribution of RC-C14 and RC-C02 in Mouse Retinal Tissue

RC-C14&GOI-E04, RC-C02&GOI-E04, and control virus AAV2&GOI-E04 were dosed via subretinal administration (4E7vg/eye) and IVT administration (2E8vg/eye) separately, 2 mice per group (6 weeks old, first group received subretinal administration (SR) in both eyes and the other group received IVT administration in both eyes, see Example 4). 4 weeks after administration, sampling was performed, sections of retinal tissue were made and immunofluorescent staining (the nucleus stained with DAPI) were performed separately.

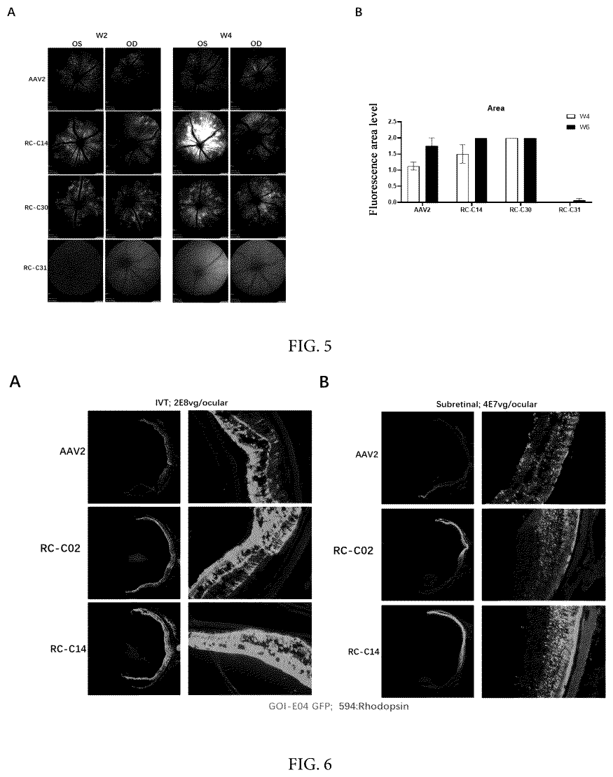

As shown in FIG. 6A, after IVT administration of RC-C14, the distribution area of retinal tissue was the widest at 4 weeks, and the photographed results from the local section also showed that the RC-C14&GOI-E04 virus has a wide distribution range in retinal tissue, and a clear green fluorescence signal was detected in all layers of the retina except for the PRE layer which was not fully covered. At the same time, the in vivo transduction activities of the above three viruses were compared by SR injection. From the panoramic and local photos of the sections in FIG. 6B, it can be found that the tissue distribution of RC-C14&GOI-E04 is significantly higher than that of RC-C02. From the (local magnified) photos of distribution in each layer of retinal tissue, it can be found that the RC-C14 virus administration group shows almost full-layer distribution, mainly distributed in the photoreceptor layer and retinal pigment epithelium. Although RC-C02 and AAV2 can also cover different layers of retinal tissue, the coverage density and fluorescence intensity are significantly weakened. Therefore, from the statistical results of IVT administration, it can be found that the RC-C14 administration group can fully penetrate the inner layer of the retina, reach the outer layer of the retina, and express the protein of interest in PR and RPE.

### Example 7: Comparison of the Distribution of RC-C14 and RC-C02 in Retinal Tissue of Cynomolgus Monkey (NHP)

The simple operation process for fluorescence photographing of the frozen sections comprises the following steps:

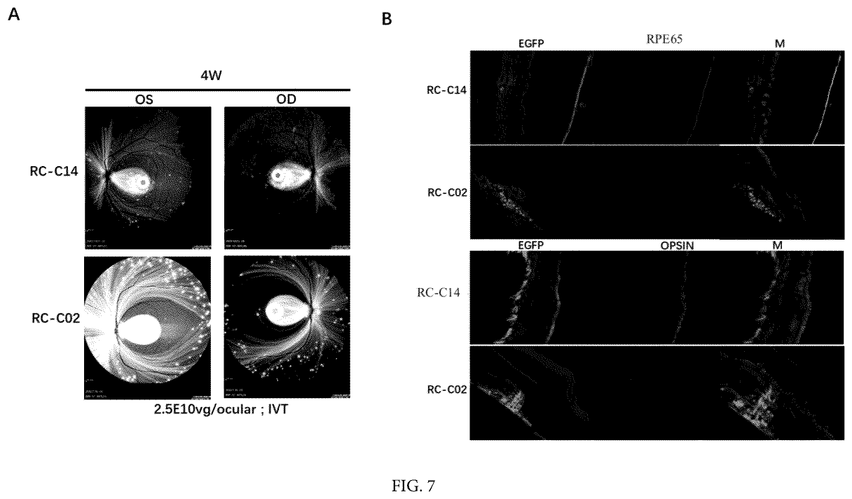

Two viruses, RC-C14 and RC-C02, were injected into eyes of 2-week-old cynomolgus monkeys (PharmaLegacy) via an IVT mode, one cynomolgus monkey per group, with a virus dose of 2.5E10vg. The administration was carried out in both eyes. 4 weeks after administration, the eyes of the monkeys are subjected to in vivo autofluorescence photographing. From the BAF fundus photography, it can be seen that the intensity and distribution of fluorescence expression of RC-C02 in the whole eye were significantly stronger than those in the RC-C14 serotype administration group (FIG. 7A).

The monkey eyes (eyeballs) were fixed in solvent for several days 4 weeks after administration, and then frozen sections were prepared. The sections with good eyeball slice morphology were selected and washed three times by soaking in PBS, 5 min each time, to remove the OCT embedding medium on the tissue surface. The tissue was circled with a histochemical pen and laid flat on a wet box. the DAPI stock solution was diluted with PBS of 1:2000 and added drop-wise on the tissue for staining for 5 min. The eye tissue was washed three times by soaking in PBS, 5 min each time, and then RPE65 (retinal pigment epithelial cells) and opsin (rhodopsin labeled photoreceptor layer) were labeled with red fluorescent secondary antibody (available from Abcam). Anti-fade fluorescence mounting medium were dropped onto the eye tissue, and a little nail polish was spotted on the edge of the slide for fixation. The slide was covered with coverslip for microscopic examination, pictures were taken through the green, blue and red fluorescence channels, and the small pictures (10×) were stitched into a complete large picture after the pictures were overlapped.

Combining the IF data statistics of the in vivo detection and retinal tissues of cynomolgus monkey, the following conclusions can be drawn: the fluorescence signal of the RC-C14 administration group was mainly concentrated in the RPE layer and the photoreceptor layer, with a stronger RPE fluorescence signal. Obvious fluorescence signals were observed in the ganglion cell layer in some areas of RC-C14 (FIG. 7B). Obvious fluorescence signals were observed in the choroidal vessels in both the RC-C14 and RC-C02 administration groups, plaque-like fluorescence signals were observed on the retinal surface, and fluorescence signals were observed from the ganglion cell layer to the inner nuclear layer in the sections. Compared with RC-C02, the RC-C14 serotype can very obviously transduce outer retinal cells such as PR and RPE.

For the inner layer RGCs, the transduction of RC-C14 is weaker than that of RC-C02; C02 has a higher transduction efficiency for the inner layer RGCs of the retina, but has limited ability to infect PR and RPE, which is also the cause of the differences in the phenomena detected by BAF.

### Example 8: Comparison of In Vitro Transduction Activity of RC-C14 and Variants Thereof

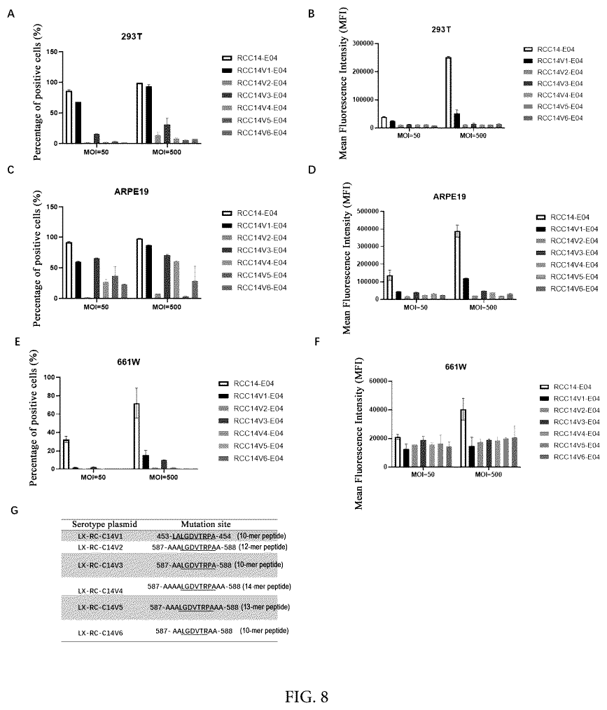

Following the same adherence method, fluorescence control viruses were prepared for RC-C14 (RCC14-E04) and 6 variants thereof (RCC14V1-E04, RCC14V2-E04, RCC14V3-E04, RCC14V4-E04, RCC14V5-E04, and RCC14V6-E04, see FIG. 8G for mutation sites), After virus purification and preparation, the physical titer of the virus was detected by qPCR, and the percentage and fluorescence intensity of green fluorescent cells in cells were detected by flow cytometry in the in vitro transduction activity experiment (see the transduction activity assay reported in Example 2 for specific methods).

FIGS. 8A-8F show statistics of the transduction activity of RC-C14, C14V1, C14V2, C14V3, C14V4, C14V5, and C14V67 serotypes in 293T, APRE19, and 661w, respectively, indicating that LALGDVTRPA 10-mer peptide can be integrated into the AAV2 VR IV variable region (RC-C14V1), exhibiting good transduction activity in 293T and ARPE19, but poor transduction activity in photoreceptor cells; with the 10-mer peptide integrated at position AAV2 VP1 587 mutated from LALGDVTRPA to AALGDVTRAA, From the transduction activity data of RC-C14V6, it can be concluded that the integration of this short peptide LALGDVTRPA in AAV variable region VIII ensures high transduction activity of the RC-C14 serotype, especially in photoreceptor cells, It was found from RC-C14V2 (12-mer peptide integrated at position 587 of VP1), RC-C14V5 (13-mer peptide integrated at position 587 of VP1), and RC-C14V4 (14-mer peptide integrated at position 587 of VP1) that the transduction activity of all the new variants was significantly reduced compared with that of RC-C14, indicating that the peptide sequence integrated in different VR regions of the AAV2 capsid is preferably a short peptide of no longer than 10 amino acids in length, which showed a close correlation to the structure formed by the inserted short peptide on the surface of the AAV capsid and the binding force to the cell surface receptor. The spatial conformation of the short peptide and the strength of the binding force between the AAV capsid and the cell surface receptor were determined by the length of the inserted peptide. Ultimately, the transduction efficiency of AAV was fed back through the reporter gene.

The sequence information is as follows:

| | SEQ ID | |
|---|---|---|
| Name | NO. | Amino acid sequence |
| AAV2 VP1 | 1 | MAADGYLPDWLEDTLSEGIRQWWKLKPGPPPPKPAERHKDDSRGL |
| | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPGKKRPVEHSPVEPDSSSGTGKAGQQPARKRLNFGQTGDA |
| | | DSVPDPQPLGQPPAAPSGLGTNTMATGSGAPMADNNEGADGVGNS |
| | | SGNWHCDSTWMGDRVITTSTRTWALPTYNNHLYKQISSQSGASND |
| | | NHYFGYSTPWGYFDFNRFHCHFSPRDWQRLINNNWGFRPKRLNFK |
| | | LFNIQVKEVTQNDGTTTIANNLTSTVQVFTDSEYQLPYVLGSAHQG |
| | | CLPPFPADVFMVPQYGYLTLNNGSQAVGRSSFYCLEYFPSQMLRTG |
| | | NNFTFSYTFEDVPFHSSYAHSQSLDRLMNPLIDQYLYYLSRTNTPSG |
| | | TTTQSRLQFSQAGASDIRDQSRNWLPGPCYRQQRVSKTSADNNNSE |
| | | YSWTGATKYHLNGRDSLVNPGPAMASHKDDEEKFFPQSGVLIFGK |
| | | QGSEKTNVDIEKVMITDEEEIRTTNPVATEQYGSVSTNLQRGNRQAA |
| | | TADVNTQGVLPGMVWQDRDVYLQGPIWAKIPHTDGHFHPSPLMG |
| | | GFGLKHPPPQILIKNTPVPANPSTTFSAAKFASFITQYSTGQVSVEIE |
| | | WELQKENSKRWNPEIQYTSNYNKSVNVDFTVDTNGVYSEPRPIGT |
| | | RYLTRNL |
| RC-C02 | 2 | MAADGYLPDWLEDTLSEGIRQWWKLKPGPPPPKPAERHKDDSRGL |
| VP1 | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPGKKRPVEHSPVEPDSSSGTGKAGQQPARKRLNFGQTGDA |
| | | DSVPDPQPLGQPPAAPSGLGTNTMATGSGAPMADNNEGADGVGNS |
| | | SGNWHCDSTWMGDRVTTTSTRTWALPTYNNHLYKQISSQSGASND |
| | | NHYFGYSTPWGYFDFNRFHCHFSPRDWQRLINNNWGFRPKRLNFK |
| | | LFNIQVKEVTQNDGTTTIANNLTSTVQVFTDSEYQLPYVLGSAHQG |
| | | CLPPFPADVFMVPQYGYLTLNNGSQAVGRSSFYCLEYFPSQMLRTG |
| | | NNFTFSYTFEDVPFHSSYAHSQSLDRLMNPLIDQYLYYLSRTNTPSG |
| | | TTTQSRLQFSQAGASDIRDQSRNWLPGPCYRQQRVSKTSADNNNSE |
| | | YSWTGATKYHLNGRDSLVNPGPAMASHKDDEEKFFPQSGVLIFGK |
| | | QGSEKTNVDIEKVMITDEEEIRTTNPVATEQYGSVSTNLQRGNLALG |
| | | ETTRPARQAATADVNTQGVLPGMVWQDRDVYLQGPIWAKIPHTDG |
| | | HFHPSPLMGGFGLKHPPPQILIKNTPVPANPSTTFSAAKFASFITQYS |
| | | TGQVSVEIEWELQKENSKRWNPEIQYTSNYNKSINVDFTVDTNGV |
| | | YSEPRPIGTRYLTRNL |
| RC-C03 | 3 | MAADGYLPDWLEDTLSEGIRQWWKLKPGPPPPKPAERHKDDSRGL |
| VP1 | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPGKKRPVEHSPVEPDSSSGTGKAGQQPARKRLNFGQTGDA |
| | | DSVPDPQPLGQPPAAPSGLGTNTMATGSGAPMADNNEGADGVGNS |
| | | SGNWHCDSTWMGDRVTTTSTRTWALPTYNNHLYKQISSQSGASND |
| | | NHYFGYSTPWGYFDFNRFHCHFSPRDWQRLINNNWGFRPKRLNFK |
| | | LFNIQVKEVTQNDGTTTIANNLTSTVQVFTDSEYQLPYVLGSAHQG |
| | | CLPPFPADVFMVPQYGYLTLNNGSQAVGRSSFYCLEYFPSQMLRTG |
| | | NNFTFSYTFEDVPFHSSYAHSQSLDRLMNPLIDQYLYFLSRTNTPSG |
| | | TTTQSRLQFSQAGASDIRDQSRNWLPGPCYRQQRVSKTSADNNNSE |
| | | YSWTGATKYHLNGRDSLVNPGPAMASHKDDEEKFFPQSGVLIFGK |
| | | QGSEKTNVDIEKVMITDEEEIRTTNPVATEQYGSVSTNLQRGNLALG |
| | | ETTRPARQAATADVNTQGVLPGMVWQDRDVYLQGPIWAKIPHTDG |
| | | HFHPSPLMGGFGLKHPPPQILIKNTPVPANPSTTFSAAKFASFITQYS |
| | | TGQVSVEIEWELQKENSKRWNPEIQYTSNYNKSINVDFTVDTNGV |
| | | YSEPRPIGTRYLTRNL |
| RC-C13 | 4 | MAADGYLPDWLEDTLSEGIRQWWKLKPGPPPPKPAERHKDDSRGL |
| VP1 | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPGKKRPVEHSPVEPDSSSGTGKAGQQPARKRLNFGQTGDA |
| | | DSVPDPQPLGQPPAAPSGLGTNTMATGSGAPMADNNEGADGVGNS |
| | | SGNWHCDSTWMGDRVTTTSTRTWALPTYNNHLYKQISSQSGASND |
| | | NHYFGYSTPWGYFDFNRFHCHFSPRDWQRLINNNWGFRPKRLNFK |
| | | LFNIQVKEVTQNDGTTTIANNLTSTVQVFTDSEYQLPYVLGSAHQG |
| | | CLPPFPADVFMVPQYGYLTLNNGSQAVGRSSFYCLEYFPSQMLRTG |
| | | NNFTFSYTFEDVPFHSSYAHSQSLDRLMNPLIDQYLYYLSRTNTPSG |
| | | TTTQSRLQFSQAGASDIRDQSRNWLPGPCYRQQRVSKTSADNNNSE |
| | | YSWTGATKYHLNGRDSLVNPGPAMASHKDDEEKFFPQSGVLIFGK |
| | | QGSEKTNVDIEKVMITDEEEIRTTNPVATEQYGSVSTNLQRGNLALG |
| | | EVTRPARQAATADVNTQGVLPGMVWQDRDVYLQGPIWAKIPHTD |
| | | GHFHPSPLMGGFGLKHPPPQILIKNTPVPANPSTTFSAAKFASFITQY |
| | | STGQVSVEIEWELQKENSKRWNPEIQYTSNYNKSINVDFTVDTNGV |
| | | YSEPRPIGTRYLTRNL |
| RC-C14 | 5 | MAADGYLPDWLEDTLSEGIRQWWKLKPGPPPPKPAERHKDDSRGL |
| VP1 | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPGKKRPVEHSPVEPDSSSGTGKAGQQPARKRLNFGQTGDA |
| | | DSVPDPQPLGQPPAAPSGLGTNTMATGSGAPMADNNEGADGVGNS |
| | | SGNWHCDSTWMGDRVTTTSTRTWALPTYNNHLYKQISSQSGASND |
| | | NHYFGYSTPWGYFDFNRFHCHFSPRDWQRLINNNWGFRPKRLNFK |
| | | LFNIQVKEVTQNDGTTTIANNLTSTVQVFTDSEYQLPYVLGSAHQG |
| | | CLPPFPADVFMVPQYGYLTLNNGSQAVGRSSFYCLEYFPSQMLRTG |
| | | NNFTFSYTFEDVPFHSSYAHSQSLDRLMNPLIDQYLYYLSRTNTPSG |
| | | TTTQSRLQFSQAGASDIRDQSRNWLPGPCYRQQRVSKTSADNNNSE |
| | | YSWTGATKYHLNGRDSLVNPGPAMASHKDDEEKFFPQSGVLIFGK |
| | | QGSEKTNVDIEKVMITDEEEIRTTNPVATEQYGSVSTNLQRGNLALG |
| | | DVTRPARQAATADVNTQGVLPGMVWQDRDVYLQGPIWAKIPHTD |
| | | GHFHPSPLMGGFGLKHPPPQILIKNTPVPANPSTTFSAAKFASFITQY |
| | | STGQVSVEIEWELQKENSKRWNPEIQYTSNYNKSINVDFTVDTNGV |
| | | YSEPRPIGTRYLTRNL |
| AAV2.7m8 | 6 | MAADGYLPDWLEDTLSEGIRQWWKLKPGPPPPKPAERHKDDSRGL |
| VP1 | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPGKKRPVEHSPVEPDSSSGTGKAGQQPARKRLNFGQTGDA |
| | | DSVPDPQPLGQPPAAPSGLGTNTMATGSGAPMADNNEGADGVGNS |
| | | SGNWHCDSTWMGDRVITTSTRTWALPTYNNHLYKQISSQSGASND |
| | | NHYFGYSTPWGYFDFNRFHCHFSPRDWQRLINNNWGFRPKRLNFK |
| | | LFNIQVKEVTQNDGTTTIANNLTSTVQVFTDSEYQLPYVLGSAHQG |
| | | CLPPFPADVFMVPQYGYLTLNNGSQAVGRSSFYCLEYFPSQMLRTG |
| | | NNFTFSYTFEDVPFHSSYAHSQSLDRLMNPLIDQYLYYLSRTNTPSG |
| | | TTTQSRLQFSQAGASDIRDQSRNWLPGPCYRQQRVSKTSADNNNSE |
| | | YSWTGATKYHLNGRDSLVNPGPAMASHKDDEEKFFPQSGVLIFGK |
| | | QGSEKTNVDIEKVMITDEEEIRTTNPVATEQYGSVSTNLQRGNLALG |
| | | ETTRPARQAATADVNTQGVLPGMVWQDRDVYLQGPIWAKIPHTDG |
| | | HFHPSPLMGGFGLKHPPPQILIKNTPVPANPSTTFSAAKFASFITQYS |
| | | TGQVSVEIEWELQKENSKRWNPEIQYTSNYNKSVNVDFTVDTNGV |
| | | YSEPRPIGTRYLTRNL |
| | SEQ ID | |
| Name | NO. | Nucleic acid sequence |
| RC-C02 | 7 | ATGGCTGCCGATGGTTATCTTCCAGATTGGCTCGAGGACACTCTC |
| VP1 | | TCTGAAGGAATAAGACAGTGGTGGAAGCTCAAACCTGGCCCACC |
| | | ACCACCAAAGCCCGCAGAGCGGCATAAGGACGACAGCAGGGGT |
| | | CTTGTGCTTCCTGGGTACAAGTACCTCGGACCCTTCAACGGACTC |
| | | GACAAGGGAGAGCCGGTCAACGAGGCAGACGCCGCGGCCCTCG |
| | | AGCACGACAAAGCCTACGACCGGCAGCTCGACAGCGGAGACAA |
| | | CCCGTACCTCAAGTACAACCACGCCGACGCGGAGTTTCAGGAGC |
| | | GCCTTAAAGAAGATACGTCTTTTGGGGGCAACCTCGGACGAGCA |
| | | GTCTTCCAGGCGAAAAAGAGGGTTCTTGAACCTCTGGGCCTGGT |
| | | TGAGGAACCTGTTAAGACGGCTCCGGGAAAAAAGAGGCCGGTA |
| | | GAGCACTCTCCTGTGGAGCCAGACTCCTCCTCGGGAACCGGAAA |
| | | GGCGGGCCAGCAGCCTGCAAGAAAAAGATTGAATTTTGGTCAGA |
| | | CTGGAGACGCAGACTCAGTACCTGACCCCCAGCCTCTCGGACAG |
| | | CCACCAGCAGCCCCCTCTGGTCTGGGAACTAATACGATGGCTAC |
| | | AGGCAGTGGCGCACCAATGGCAGACAATAACGAGGGCGCCGAC |
| | | GGAGTGGGTAATTCCTCGGGAAATTGGCATTGCGATTCCACATGG |
| | | ATGGGCGACAGAGTCAcCACCACCAGCACCCGAACCTGGGCCCT |
| | | GCCCACCTACAACAACCACCTCTACAAACAAATTTCCAGCCAAT |
| | | CAGGAGCCTCGAACGACAATCACTACTTTGGCTACAGCACCCCT |
| | | TGGGGGTATTTTGACTTCAACAGATTCCACTGCCACTTTTCACCA |
| | | CGTGACTGGCAAAGACTCATCAACAACAACTGGGGATTCCGACC |
| | | CAAGAGACTCAACTTCAAGCTCTTTAACATTCAAGTCAAAGAGG |
| | | TCACGCAGAATGACGGTACGACGACGATTGCCAATAACCTTACC |
| | | AGCACGGTTCAGGTGTTTACTGACTCGGAGTACCAGCTCCCGTA |
| | | CGTCCTCGGCTCGGCGCATCAAGGATGCCTCCCGCCGTTCCCAG |
| | | CAGACGTCTTCATGGTGCCACAGTATGGATACCTCACCCTGAACA |
| | | ACGGGAGTCAGGCAGTAGGACGCTCTTCATTTTACTGCCTGGAGT |
| | | ACTTTCCTTCTCAGATGCTGCGTACCGGAAACAACTTTACCTTCA |
| | | GCTACACTTTTGAGGACGTTCCTTTCCACAGCAGCTACGCTCACA |
| | | GCCAGAGTCTGGACCGTCTCATGAATCCTCTCATCGACCAGTACC |
| | | TGTATTACTTGAGCAGAACAAACACTCCAAGTGGAACCACCACG |
| | | CAGTCAAGGCTTCAGTTTTCTCAGGCCGGAGCGAGTGACATTCG |
| | | GGACCAGTCTAGGAACTGGCTTCCTGGACCCTGTTACCGCCAGC |
| | | AGCGAGTATCAAAGACATCTGCGGATAACAACAACAGTGAATAC |
| | | TCGTGGACTGGAGCTACCAAGTACCACCTCAATGGCAGAGACTC |
| | | TCTGGTGAATCCGGGCCCGGCCATGGCAAGCCACAAGGACGATG |
| | | AAGAAAAGTTTTTTCCTCAGAGCGGGGTTCTCATCTTTGGGAAG |
| | | CAAGGCTCAGAGAAAACAAATGTGGACATTGAAAAGGTCATGAT |
| | | TACAGACGAAGAGGAAATCAGGACAACCAATCCCGTGGCTACGG |
| | | AGCAGTATGGTTCTGTATCTACCAACCTCCAGAGAGGCAACCTAG |
| | | CACTCGGCGAAACAACAAGACCTGCTAGGCAAGCAGCTACCGC |
| | | AGATGTCAACACACAAGGCGTTCTTCCAGGCATGGTCTGGCAGG |
| | | ACAGAGATGTGTACCTTCAGGGGCCCATCTGGGCAAAGATTCCA |
| | | CACACGGACGGACATTTTCACCCCTCTCCCCTCATGGGTGGATTC |
| | | GGACTTAAACACCCTCCTCCACAGATTCTCATCAAGAACACCCC |
| | | GGTACCTGCGAATCCTTCGACCACCTTCAGTGCGGCAAAGTTTG |
| | | CTTCCTTCATCACACAGTACTCCACGGGACAGGTCAGCGTGGAG |
| | | ATCGAGTGGGAGCTGCAGAAGGAAAACAGCAAACGCTGGAATC |
| | | CCGAAATTCAGTACACTTCCAACTACAACAAGTCTaTTAATGTGG |
| | | ACTTTACTGTGGACACTAATGGCGTGTATTCAGAGCCTCGCCCCA |
| | | TTGGCACCAGATACCTGACTCGTAATCTGTAA |
| RC-C03 | 8 | ATGGCTGCCGATGGTTATCTTCCAGATTGGCTCGAGGACACTCTC |
| VP1 | | TCTGAAGGAATAAGACAGTGGTGGAAGCTCAAACCTGGCCCACC |
| | | ACCACCAAAGCCCGCAGAGCGGCATAAGGACGACAGCAGGGGT |
| | | CTTGTGCTTCCTGGGTACAAGTACCTCGGACCCTTCAACGGACTC |
| | | GACAAGGGAGAGCCGGTCAACGAGGCAGACGCCGCGGCCCTCG |
| | | AGCACGACAAAGCCTACGACCGGCAGCTCGACAGCGGAGACAA |
| | | CCCGTACCTCAAGTACAACCACGCCGACGCGGAGTTTCAGGAGC |
| | | GCCTTAAAGAAGATACGTCTTTTGGGGGCAACCTCGGACGAGCA |
| | | GTCTTCCAGGCGAAAAAGAGGGTTCTTGAACCTCTGGGCCTGGT |
| | | TGAGGAACCTGTTAAGACGGCTCCGGGAAAAAAGAGGCCGGTA |
| | | GAGCACTCTCCTGTGGAGCCAGACTCCTCCTCGGGAACCGGAAA |
| | | GGCGGGCCAGCAGCCTGCAAGAAAAAGATTGAATTTTGGTCAGA |
| | | CTGGAGACGCAGACTCAGTACCTGACCCCCAGCCTCTCGGACAG |
| | | CCACCAGCAGCCCCCTCTGGTCTGGGAACTAATACGATGGCTAC |
| | | AGGCAGTGGCGCACCAATGGCAGACAATAACGAGGGCGCCGAC |
| | | GGAGTGGGTAATTCCTCGGGAAATTGGCATTGCGATTCCACATGG |
| | | ATGGGCGACAGAGTCAcCACCACCAGCACCCGAACCTGGGCCCT |
| | | GCCCACCTACAACAACCACCTCTACAAACAAATTTCCAGCCAAT |
| | | CAGGAGCCTCGAACGACAATCACTACTTTGGCTACAGCACCCCT |
| | | TGGGGGTATTTTGACTTCAACAGATTCCACTGCCACTTTTCACCA |
| | | CGTGACTGGCAAAGACTCATCAACAACAACTGGGGATTCCGACC |
| | | CAAGAGACTCAACTTCAAGCTCTTTAACATTCAAGTCAAAGAGG |
| | | TCACGCAGAATGACGGTACGACGACGATTGCCAATAACCTTACC |
| | | AGCACGGTTCAGGTGTTTACTGACTCGGAGTACCAGCTCCCGTA |
| | | CGTCCTCGGCTCGGCGCATCAAGGATGCCTCCCGCCGTTCCCAG |
| | | CAGACGTCTTCATGGTGCCACAGTATGGATACCTCACCCTGAACA |
| | | ACGGGAGTCAGGCAGTAGGACGCTCTTCATTTTACTGCCTGGAGT |
| | | ACTTTCCTTCTCAGATGCTGCGTACCGGAAACAACTTTACCTTCA |
| | | GCTACACTTTTGAGGACGTTCCTTTCCACAGCAGCTACGCTCACA |
| | | GCCAGAGTCTGGACCGTCTCATGAATCCTCTCATCGACCAGTACC |
| | | TGTATTtCTTGAGCAGAACAAACACTCCAAGTGGAACCACCACGC |
| | | AGTCAAGGCTTCAGTTTTCTCAGGCCGGAGCGAGTGACATTCGG |
| | | GACCAGTCTAGGAACTGGCTTCCTGGACCCTGTTACCGCCAGCA |
| | | GCGAGTATCAAAGACATCTGCGGATAACAACAACAGTGAATACT |
| | | CGTGGACTGGAGCTACCAAGTACCACCTCAATGGCAGAGACTCT |
| | | CTGGTGAATCCGGGCCCGGCCATGGCAAGCCACAAGGACGATGA |
| | | AGAAAAGTTTTTTCCTCAGAGCGGGGTTCTCATCTTTGGGAAGC |
| | | AAGGCTCAGAGAAAACAAATGTGGACATTGAAAAGGTCATGATT |
| | | ACAGACGAAGAGGAAATCAGGACAACCAATCCCGTGGCTACGG |
| | | AGCAGTATGGTTCTGTATCTACCAACCTCCAGAGAGGCAACCTAG |
| | | CACTCGGCGAAACAACAAGACCTGCTAGGCAAGCAGCTACCGC |
| | | AGATGTCAACACACAAGGCGTTCTTCCAGGCATGGTCTGGCAGG |
| | | ACAGAGATGTGTACCTTCAGGGGCCCATCTGGGCAAAGATTCCA |
| | | CACACGGACGGACATTTTCACCCCTCTCCCCTCATGGGTGGATTC |
| | | GGACTTAAACACCCTCCTCCACAGATTCTCATCAAGAACACCCC |
| | | GGTACCTGCGAATCCTTCGACCACCTTCAGTGCGGCAAAGTTTG |
| | | CTTCCTTCATCACACAGTACTCCACGGGACAGGTCAGCGTGGAG |
| | | ATCGAGTGGGAGCTGCAGAAGGAAAACAGCAAACGCTGGAATC |
| | | CCGAAATTCAGTACACTTCCAACTACAACAAGTCTaTTAATGTGG |
| | | ACTTTACTGTGGACACTAATGGCGTGTATTCAGAGCCTCGCCCCA |
| | | TTGGCACCAGATACCTGACTCGTAATCTGTAA |
| RC-C13 | 9 | ATGGCTGCCGATGGTTATCTTCCAGATTGGCTCGAGGACACTCTC |
| VP1 | | TCTGAAGGAATAAGACAGTGGTGGAAGCTCAAACCTGGCCCACC |
| | | ACCACCAAAGCCCGCAGAGCGGCATAAGGACGACAGCAGGGGT |
| | | CTTGTGCTTCCTGGGTACAAGTACCTCGGACCCTTCAACGGACTC |
| | | GACAAGGGAGAGCCGGTCAACGAGGCAGACGCCGCGGCCCTCG |
| | | AGCACGACAAAGCCTACGACCGGCAGCTCGACAGCGGAGACAA |
| | | CCCGTACCTCAAGTACAACCACGCCGACGCGGAGTTTCAGGAGC |
| | | GCCTTAAAGAAGATACGTCTTTTGGGGGCAACCTCGGACGAGCA |
| | | GTCTTCCAGGCGAAAAAGAGGGTTCTTGAACCTCTGGGCCTGGT |
| | | TGAGGAACCTGTTAAGACGGCTCCGGGAAAAAAGAGGCCGGTA |
| | | GAGCACTCTCCTGTGGAGCCAGACTCCTCCTCGGGAACCGGAAA |
| | | GGCGGGCCAGCAGCCTGCAAGAAAAAGATTGAATTTTGGTCAGA |
| | | CTGGAGACGCAGACTCAGTACCTGACCCCCAGCCTCTCGGACAG |
| | | CCACCAGCAGCCCCCTCTGGTCTGGGAACTAATACGATGGCTAC |
| | | AGGCAGTGGCGCACCAATGGCAGACAATAACGAGGGCGCCGAC |
| | | GGAGTGGGTAATTCCTCGGGAAATTGGCATTGCGATTCCACATGG |
| | | ATGGGCGACAGAGTCAcCACCACCAGCACCCGAACCTGGGCCCT |
| | | GCCCACCTACAACAACCACCTCTACAAACAAATTTCCAGCCAAT |
| | | CAGGAGCCTCGAACGACAATCACTACTTTGGCTACAGCACCCCT |
| | | TGGGGGTATTTTGACTTCAACAGATTCCACTGCCACTTTTCACCA |
| | | CGTGACTGGCAAAGACTCATCAACAACAACTGGGGATTCCGACC |
| | | CAAGAGACTCAACTTCAAGCTCTTTAACATTCAAGTCAAAGAGG |
| | | TCACGCAGAATGACGGTACGACGACGATTGCCAATAACCTTACC |
| | | AGCACGGTTCAGGTGTTTACTGACTCGGAGTACCAGCTCCCGTA |
| | | CGTCCTCGGCTCGGCGCATCAAGGATGCCTCCCGCCGTTCCCAG |
| | | CAGACGTCTTCATGGTGCCACAGTATGGATACCTCACCCTGAACA |
| | | ACGGGAGTCAGGCAGTAGGACGCTCTTCATTTTACTGCCTGGAGT |
| | | ACTTTCCTTCTCAGATGCTGCGTACCGGAAACAACTTTACCTTCA |
| | | GCTACACTTTTGAGGACGTTCCTTTCCACAGCAGCTACGCTCACA |
| | | GCCAGAGTCTGGACCGTCTCATGAATCCTCTCATCGACCAGTACC |
| | | TGTATTACTTGAGCAGAACAAACACTCCAAGTGGAACCACCACG |
| | | CAGTCAAGGCTTCAGTTTTCTCAGGCCGGAGCGAGTGACATTCG |
| | | GGACCAGTCTAGGAACTGGCTTCCTGGACCCTGTTACCGCCAGC |
| | | AGCGAGTATCAAAGACATCTGCGGATAACAACAACAGTGAATAC |
| | | TCGTGGACTGGAGCTACCAAGTACCACCTCAATGGCAGAGACTC |
| | | TCTGGTGAATCCGGGCCCGGCCATGGCAAGCCACAAGGACGATG |
| | | AAGAAAAGTTTTTTCCTCAGAGCGGGGTTCTCATCTTTGGGAAG |
| | | CAAGGCTCAGAGAAAACAAATGTGGACATTGAAAAGGTCATGAT |
| | | TACAGACGAAGAGGAAATCAGGACAACCAATCCCGTGGCTACGG |
| | | AGCAGTATGGTTCTGTATCTACCAACCTCCAGAGAGGCAACCTAG |
| | | CACTCGGCGAAgtAACAAGACCTGCTAGGCAAGCAGCTACCGCA |
| | | GATGTCAACACACAAGGCGTTCTTCCAGGCATGGTCTGGCAGGA |
| | | CAGAGATGTGTACCTTCAGGGGCCCATCTGGGCAAAGATTCCAC |
| | | ACACGGACGGACATTTTCACCCCTCTCCCCTCATGGGTGGATTCG |
| | | GACTTAAACACCCTCCTCCACAGATTCTCATCAAGAACACCCCG |
| | | GTACCTGCGAATCCTTCGACCACCTTCAGTGCGGCAAAGTTTGCT |
| | | TCCTTCATCACACAGTACTCCACGGGACAGGTCAGCGTGGAGAT |
| | | CGAGTGGGAGCTGCAGAAGGAAAACAGCAAACGCTGGAATCCC |
| | | GAAATTCAGTACACTTCCAACTACAACAAGTCTaTTAATGTGGAC |
| | | TTTACTGTGGACACTAATGGCGTGTATTCAGAGCCTCGCCCCATT |
| | | GGCACCAGATACCTGACTCGTAATCTGTAA |
| RC-C14 | 10 | ATGGCTGCCGATGGTTATCTTCCAGATTGGCTCGAGGACACTCTC |
| VP1 | | TCTGAAGGAATAAGACAGTGGTGGAAGCTCAAACCTGGCCCACC |
| | | ACCACCAAAGCCCGCAGAGCGGCATAAGGACGACAGCAGGGGT |
| | | CTTGTGCTTCCTGGGTACAAGTACCTCGGACCCTTCAACGGACTC |
| | | GACAAGGGAGAGCCGGTCAACGAGGCAGACGCCGCGGCCCTCG |
| | | AGCACGACAAAGCCTACGACCGGCAGCTCGACAGCGGAGACAA |
| | | CCCGTACCTCAAGTACAACCACGCCGACGCGGAGTTTCAGGAGC |
| | | GCCTTAAAGAAGATACGTCTTTTGGGGGCAACCTCGGACGAGCA |
| | | GTCTTCCAGGCGAAAAAGAGGGTTCTTGAACCTCTGGGCCTGGT |
| | | TGAGGAACCTGTTAAGACGGCTCCGGGAAAAAAGAGGCCGGTA |
| | | GAGCACTCTCCTGTGGAGCCAGACTCCTCCTCGGGAACCGGAAA |
| | | GGCGGGCCAGCAGCCTGCAAGAAAAAGATTGAATTTTGGTCAGA |
| | | CTGGAGACGCAGACTCAGTACCTGACCCCCAGCCTCTCGGACAG |
| | | CCACCAGCAGCCCCCTCTGGTCTGGGAACTAATACGATGGCTAC |
| | | AGGCAGTGGCGCACCAATGGCAGACAATAACGAGGGCGCCGAC |
| | | GGAGTGGGTAATTCCTCGGGAAATTGGCATTGCGATTCCACATGG |
| | | ATGGGCGACAGAGTCAcCACCACCAGCACCCGAACCTGGGCCCT |
| | | GCCCACCTACAACAACCACCTCTACAAACAAATTTCCAGCCAAT |
| | | CAGGAGCCTCGAACGACAATCACTACTTTGGCTACAGCACCCCT |
| | | TGGGGGTATTTTGACTTCAACAGATTCCACTGCCACTTTTCACCA |
| | | CGTGACTGGCAAAGACTCATCAACAACAACTGGGGATTCCGACC |
| | | CAAGAGACTCAACTTCAAGCTCTTTAACATTCAAGTCAAAGAGG |
| | | TCACGCAGAATGACGGTACGACGACGATTGCCAATAACCTTACC |
| | | AGCACGGTTCAGGTGTTTACTGACTCGGAGTACCAGCTCCCGTA |
| | | CGTCCTCGGCTCGGCGCATCAAGGATGCCTCCCGCCGTTCCCAG |
| | | CAGACGTCTTCATGGTGCCACAGTATGGATACCTCACCCTGAACA |
| | | ACGGGAGTCAGGCAGTAGGACGCTCTTCATTTTACTGCCTGGAGT |
| | | ACTTTCCTTCTCAGATGCTGCGTACCGGAAACAACTTTACCTTCA |
| | | GCTACACTTTTGAGGACGTTCCTTTCCACAGCAGCTACGCTCACA |
| | | GCCAGAGTCTGGACCGTCTCATGAATCCTCTCATCGACCAGTACC |
| | | TGTATTACTTGAGCAGAACAAACACTCCAAGTGGAACCACCACG |
| | | CAGTCAAGGCTTCAGTTTTCTCAGGCCGGAGCGAGTGACATTCG |
| | | GGACCAGTCTAGGAACTGGCTTCCTGGACCCTGTTACCGCCAGC |
| | | AGCGAGTATCAAAGACATCTGCGGATAACAACAACAGTGAATAC |
| | | TCGTGGACTGGAGCTACCAAGTACCACCTCAATGGCAGAGACTC |
| | | TCTGGTGAATCCGGGCCCGGCCATGGCAAGCCACAAGGACGATG |
| | | AAGAAAAGTTTTTTCCTCAGAGCGGGGTTCTCATCTTTGGGAAG |
| | | CAAGGCTCAGAGAAAACAAATGTGGACATTGAAAAGGTCATGAT |
| | | TACAGACGAAGAGGAAATCAGGACAACCAATCCCGTGGCTACGG |
| | | AGCAGTATGGTTCTGTATCTACCAACCTCCAGAGAGGCAACCTAG |
| | | CACTCGGCGATGTAACAAGACCTGCTAGGCAAGCAGCTACCGCA |
| | | GATGTCAACACACAAGGCGTTCTTCCAGGCATGGTCTGGCAGGA |
| | | CAGAGATGTGTACCTTCAGGGGCCCATCTGGGCAAAGATTCCAC |
| | | ACACGGACGGACATTTTCACCCCTCTCCCCTCATGGGTGGATTCG |
| | | GACTTAAACACCCTCCTCCACAGATTCTCATCAAGAACACCCCG |
| | | GTACCTGCGAATCCTTCGACCACCTTCAGTGCGGCAAAGTTTGCT |
| | | TCCTTCATCACACAGTACTCCACGGGACAGGTCAGCGTGGAGAT |
| | | CGAGTGGGAGCTGCAGAAGGAAAACAGCAAACGCTGGAATCCC |
| | | GAAATTCAGTACACTTCCAACTACAACAAGTCTaTTAATGTGGAC |
| | | TTTACTGTGGACACTAATGGCGTGTATTCAGAGCCTCGCCCCATT |
| | | GGCACCAGATACCTGACTCGTAATCTGTAA |
| RC-C02-F1 | 11 | AACAATAAATGATTTAAATCAGGTATGG |
| RC-C02-R1 | 12 | GTGGTGGTGACTCTGTCGCCCATCCATG |
| RC-C02-F2 | 13 | ACAGAGTCACCACCACCAGCACCCGAACC |
| RC-C02-R2 | 14 | TTGTTGTTTCGCCGAGTGCTAGGTTGCCTCTCTGGAGGTTG |
| RC-C02-F3 | 15 | TCGGCGAAACAACAAGACCTGCTAGGCAAGCAGCTACCGCAG |
| RC-C02-R3 | 16 | ACATTAATAGACTTGTTGTAGTTGGAAG |
| RC-C02-F4 | 17 | ACAAGTCTATTAATGTGGACTTTACTGTGG |
| RC-C02-R4 | 18 | GCTGTTTAAACGCCCGGGCTGTAG |
| RC-C03-R1 | 19 | GCTCAAGAAATACAGGTACTGGTCGATG |
| RC-C03-F2 | 20 | CCTGTATTTCTTGAGCAGAACAAACACTC |
| RC-C13-R1 | 21 | GTCTTGTTACTTCGCCGAGTGCTAGGTTG |
| RC-C13-F2 | 22 | CACTCGGCGAAGTAACAAGACCTGCTAGGCAAG |
| RC-C14-R1 | 23 | TCTTGTTACATCGCCGAGTGCTAGGTTGC |
| RC-C14-F2 | 24 | TCGGCGATGTAACAAGACCTGCTAGGCAAG |
| GOI-E04 | 25 | AGCGCCCAATACGCAAACCGCCTCTCCCCGCGCGTTGGCCGATT |
| Plasmid | | CATTAATGCAGCTGGCACGACAGGTTTCCCGACTGGAAAGCGG |
| sequence | | GCAGTGAGCGCAACGCAATTAATGTGAGTTAGCTCACTCATTAG |
| | | GCACCCCAGGCTTTACACTTTATGCTTCCGGCTCGTATGTTGTGT |
| | | GGAATTGTGAGCGGATAACAATTTCACACAGGAAACAGCTATG |
| | | ACCATGATTACGAATTGCCTGCAGGCAGCTGCGCGCTCGCTCGC |
| | | TCACTGAGGCCGCCCGGGCAAAGCCCGGGCGTCGGGCGACCTTT |
| | | GGTCGCCCGGCCTCAGTGAGCGAGCGAGCGCGCAGAGAGGGAG |
| | | TGGCCAACTCCATCACTAGGGGTTCCTATCGATATCAAGCTTCG |
| | | TTACATAACTTACGGTAAATGGCCCGCCTGGCTGACCGCCCAAC |
| | | GACCCCCGCCCATTGACGTCAATAATGACGTATGTTCCCATAGT |
| | | AACGCCAATAGGGACTTTCCATTGACGTCAATGGGTGGACTATT |
| | | TACGGTAAACTGCCCACTTGGCAGTACATCAAGTGTATCATATG |
| | | CCAAGTACGCCCCCTATTGACGTCAATGACGGTAAATGGCCCGC |
| | | CTGGCATTATGCCCAGTACATGACCTTATGGGACTTTCCTACTTG |
| | | GCAGTACATCTACGTATTAGTCATCGCTATTACCATGGGTCGAG |
| | | GTGAGCCCCACGTTCTGCTTCACTCTCCCCATCTCCCCCCCCTCC |
| | | CCACCCCCAATTTTGTATTTATTTATTTTTTAATTATTTTGTGCAG |
| | | CGATGGGGGCGGGGGGGGGGGGGGCGCGCGCCAGGCGGGGCG |
| | | GGGCGGGGCGAGGGGGGGGCGGGGCGAGGCGGAGAGGTGCG |
| | | GCGGCAGCCAATCAGAGCGGCGCGCTCCGAAAGTTTCCTTTTAT |
| | | GGCGAGGCGGCGGCGGCGGCGGCCCTATAAAAAGCGAAGCGCG |
| | | CGGCGGGCGGATATCGGAGTCGCTGCGTTGCCTTCGCCCCGTGC |
| | | CCCGCTCCGCGCCGCCTCGCGCCGCCCGCCCCGGCTCTGACTGA |
| | | CCGCGTTACTCCCACAGGTGAGCGGGCGGGACGGCCCTTCTCCT |
| | | CCGGGCTGTAATTAGCGCTTGGTTTAATGACGGCTCGTTTCTTTT |
| | | CTGTGGCTGCGTGAAAGCCTTAAAGGGCTCCGGGAGGGCCCTTT |
| | | GTGCGGGGGGGAGCGGCTCGGGGGGTGCGTGCGTGTGTGTGTG |
| | | CGTGGGGAGCGCCGCGTGCGGCCCGCGCTGCCCGGCGGCTGTG |
| | | AGCGCTGCGGGCGCGGCGCGGGGCTTTGTGCGCTCCGCGTGTGC |
| | | GCGAGGGGAGCGCGGCCGGGGGCGGTGCCCCGCGGTGCGGGGG |
| | | GGCTGCGAGGGGAACAAAGGCTGCGTGCGGGGTGTGTGCGTGG |
| | | GGGGGTGAGCAGGGGGTGTGGGCGCGGCGGTCGGGCTGTAACC |
| | | CCCCCCTGCACCCCCCTCCCCGAGTTGCTGAGCACGGCCCGGCT |
| | | TCGGGTGCGGGGCTCCGTGCGGGGCGTGGCGCGGGGCTCGCCG |
| | | TGCCGGGCGGGGGGTGGCGGCAGGTGGGGGTGCCGGGCGGGGC |
| | | GGGGCCGCCTCGGGCCGGGGAGGGCTCGGGGGAGGGGCGCGGC |
| | | GGCCCCGGAGCGCCGGCGGCTGTCGAGGCGCGGCGAGCCGCAG |
| | | CCATTGCCTTTTATGGTAATCGTGCGAGAGGGCGCAGGGACTTC |
| | | CTTTGTCCCAAATCTGGCGGAGCCGAAATCTGGGAGGCGCCGCC |
| | | GCACCCCCTCTAGCGGGCGCGGGCGAAGCGGTGCGGCGCCGGC |
| | | AGGAAGGAAATGGGCGGGGAGGGCCTTCGTGCGTCGCCGCGCC |
| | | GCCGTCCCCTTCTCCATCTCCAGCCTCGGGGCTGCCGCAGGGGG |
| | | ACGGCTGCCTTCGGGGGGGACGGGGCAGGGCGGGGTTCGGCTT |
| | | CTGGCGTGTGACCGGCGGCTCTAGAGCCTCTGCTAACCATGTTC |
| | | ATGCCTTCTTCTTTTTCCTACAGGGGCCCAGCCGGCCTCGCGAG |
| | | AATTCTCTAGAACGCCGCCATGGTGAGCAAGGGCGAGGAGCTG |
| | | TTCACCGGGGTGGTGCCCATCCTGGTCGAGCTGGACGGCGACGT |
| | | AAACGGCCACAAGTTCAGCGTGTCCGGCGAGGGCGAGGGCGAT |
| | | GCCACCTACGGCAAGCTGACCCTGAAGTTCATCTGCACCACCGG |
| | | CAAGCTGCCCGTGCCCTGGCCCACCCTCGTGACCACCCTGACCT |
| | | ACGGCGTGCAGTGCTTCAGCCGCTACCCCGACCACATGAAGCAG |
| | | CACGACTTCTTCAAGTCCGCCATGCCCGAAGGCTACGTCCAGGA |
| | | GCGCACCATCTTCTTCAAGGACGACGGCAACTACAAGACCCGCG |
| | | CCGAGGTGAAGTTCGAGGGCGACACCCTGGTGAACCGCATCGA |
| | | GCTGAAGGGCATCGACTTCAAGGAGGACGGCAACATCCTGGGG |
| | | CACAAGCTGGAGTACAACTACAACAGCCACAACGTCTATATCAT |
| | | GGCCGACAAGCAGAAGAACGGCATCAAGGTGAACTTCAAGATC |
| | | CGCCACAACATCGAGGACGGCAGCGTGCAGCTCGCCGACCACT |
| | | ACCAGCAGAACACCCCCATCGGCGACGGCCCCGTGCTGCTGCCC |
| | | GACAACCACTACCTGAGCACCCAGTCCGCCCTGAGCAAAGACC |
| | | CCAACGAGAAGCGCGATCACATGGTCCTGCTGGAGTTCGTGACC |
| | | GCCGCCGGGATCACTCTCGGCATGGACGAGCTGTACAAGTAGA |
| | | GCTGACACTAGTGCGGATCCACGGGTGGCATCCCTGTGACCCCT |
| | | CCCCAGTGCCTCTCCTGGCCCTGGAAGTTGCCACTCCAGTGCCC |
| | | ACCAGCCTTGTCCTAATAAAATTAAGTTGCATCATTTTGTCTGAC |
| | | TAGGTGTCCTTCTATAATATTATGGGGTGGAGGGGGGTGGTATG |
| | | GAGCAAGGGGCAAGTTGGGAAGACAACCTGTAGGGCCTGCGGG |
| | | GTCTATTGGGAACCAAGCTGGAGTGCAGTGGCACAATCTTGGCT |
| | | CACTGCAATCTCCGCCTCCTGGGTTCAAGCGATTCTCCTGCCTCA |
| | | GCCTCCCGAGTTGTTGGGATTCCAGGCATGCATGACCAGGCTCA |
| | | GCTAATTTTTGTTTTTTTGGTAGAGACGGGGTTTCACCATATTGG |
| | | CCAGGCTGGTCTCCAACTCCTAATCTCAGGTGATCTACCCACCTT |
| | | GGCCTCCCAAATTGCTGGGATTACAGGCGTGAACCACTGCTCCC |
| | | TTCCCTGTCCTTATCGATAGATCTAGGAACCCCTAGTGATGGAG |
| | | TTGGCCACTCCCTCTCTGCGCGCTCGCTCGCTCACTGAGGCCGG |
| | | GCGACCAAAGGTCGCCCGACGCCCGGGCTTTGCCCGGGCGGCCT |
| | | CAGTGAGCGAGCGAGCGCGCAGCTGCCTGCAGGCAGCTTGGCA |
| | | CTGGCCGTCGTTTTACAACGTCGTGACTGGGAAAACCCTGGCGT |
| | | TACCCAACTTAATCGCCTTGCAGCACATCCCCCTTTCGCCAGCTG |
| | | GCGTAATAGCGAAGAGGCCCGCACCGATCGCCCTTCCCAACAGT |
| | | TGCGCAGCCTGAATGGCGAATGGCGCCTGATGCGGTATTTTCTC |
| | | CTTACGCATCTGTGCGGTATTTCACACCGCATACGTCAAAGCAA |
| | | CCATAGTACGCGCCCTGTAGCGGCGCATTAAGCGCGGCGGGTGT |
| | | GGTGGTTACGCGCAGCGTGACCGCTACACTTGCCAGCGCCCTAG |
| | | CGCCCGCTCCTTTCGCTTTCTTCCCTTCCTTTCTCGCCACGTTCGC |
| | | CGGCTTTCCCCGTCAAGCTCTAAATCGGGGGCTCCCTTTAGGGT |
| | | TCCGATTTAGTGCTTTACGGCACCTCGACCCCAAAAAACTTGAT |
| | | TTGGGTGATGGTTCACGTAGTGGGCCATCGCCCTGATAGACGGT |
| | | TTTTCGCCCTTTGACGTTGGAGTCCACGTTCTTTAATAGTGGACT |
| | | CTTGTTCCAAACTGGAACAACACTCAACCCTATCTCGGGCTATT |
| | | CTTTTGATTTATAAGGGATTTTGCCGATTTCGGCCTATTGGTTAA |
| | | AAAATGAGCTGATTTAACAAAAATTTAACGCGAATTTTAACAAA |
| | | ATATTAACGTTTACAATTTTATGGTGCACTCTCAGTACAATCTGC |
| | | TCTGATGCCGCATAGTTAAGCCAGCCCCGACACCCGCCAACACC |
| | | CGCTGACGCGCCCTGACGGGCTTGTCTGCTCCCGGCATCCGCTT |
| | | ACAGACAAGCTGTGACCGTCTCCGGGAGCTGCATGTGTCAGAG |
| | | GTTTTCACCGTCATCACCGAAACGCGCGAGACGAAAGGGCCTCG |
| | | TGATACGCCTATTTTTATAGGTTAATGTCATGATAATAATGGTTT |
| | | CTTAGACGTCAGGTGGCACTTTTCGGGGAAATGTGCGGAACCCC |
| | | TATTTGTTTATTTTTCTAAATACATTCAAATATGTATCCGCTCAT |
| | | GAATTAATTCTTAGAAAAACTCATCGAGCATCAAATGAAACTGC |
| | | AATTTATTCATATCAGGATTATCAATACCATATTTTTGAAAAAG |
| | | CCGTTTCTGTAATGAAGGAGAAAACTCACCGAGGCAGTTCCATA |
| | | GGATGGCAAGATCCTGGTATCGGTCTGCGATTCCGACTCGTCCA |
| | | ACATCAATACAACCTATTAATTTCCCCTCGTCAAAAATAAGGTT |
| | | ATCAAGTGAGAAATCACCATGAGTGACGACTGAATCCGGTGAG |
| | | AATGGCAAAAGTTTATGCATTTCTTTCCAGACTTGTTCAACAGG |
| | | CCAGCCATTACGCTCGTCATCAAAATCACTCGCATCAACCAAAC |
| | | CGTTATTCATTCGTGATTGCGCCTGAGCGAGACGAAATACGCGA |
| | | TCGCTGTTAAAAGGACAATTACAAACAGGAATCGAATGCAACC |
| | | GGCGCAGGAACACTGCCAGCGCATCAACAATATTTTCACCTGAA |
| | | TCAGGATATTCTTCTAATACCTGGAATGCTGTTTTCCCGGGGATC |
| | | GCAGTGGTGAGTAACCATGCATCATCAGGAGTACGGATAAAAT |
| | | GCTTGATGGTCGGAAGAGGCATAAATTCCGTCAGCCAGTTTAGT |
| | | CTGACCATCTCATCTGTAACATCATTGGCAACGCTACCTTTGCCA |
| | | TGTTTCAGAAACAACTCTGGCGCATCGGGCTTCCCATACAATCG |
| | | ATAGATTGTCGCACCTGATTGCCCGACATTATCGCGAGCCCATT |
| | | TATACCCATATAAATCAGCATCCATGTTGGAATTTAATCGCGGC |
| | | CTAGAGCAAGACGTTTCCCGTTGAATATGGCTCATAACACCCCT |
| | | TGTATTACTGTTTATGTAAGCAGACAGTTTTATTGTTCATGACCA |
| | | AAATCCCTTAACGTGAGTTTTCGTTCCACTGAGCGTCAGACCCC |
| | | GTAGAAAAGATCAAAGGATCTTCTTGAGATCCTTTTTTTCTGCG |
| | | CGTAATCTGCTGCTTGCAAACAAAAAAACCACCGCTACCAGCGG |
| | | TGGTTTGTTTGCCGGATCAAGAGCTACCAACTCTTTTTCCGAAG |
| | | GTAACTGGCTTCAGCAGAGCGCAGATACCAAATACTGTTCTTCT |
| | | AGTGTAGCCGTAGTTAGGCCACCACTTCAAGAACTCTGTAGCAC |
| | | CGCCTACATACCTCGCTCTGCTAATCCTGTTACCAGTGGCTGCTG |
| | | CCAGTGGCGATAAGTCGTGTCTTACCGGGTTGGACTCAAGACGA |
| | | TAGTTACCGGATAAGGCGCAGCGGTCGGGCTGAACGGGGGGTT |
| | | CGTGCACACAGCCCAGCTTGGAGCGAACGACCTACACCGAACT |
| | | GAGATACCTACAGCGTGAGCTATGAGAAAGCGCCACGCTTCCC |
| | | GAAGGGAGAAAGGCGGACAGGTATCCGGTAAGCGGCAGGGTCG |
| | | GAACAGGAGAGCGCACGAGGGAGCTTCCAGGGGGAAACGCCTG |
| | | GTATCTTTATAGTCCTGTCGGGTTTCGCCACCTCTGACTTGAGCG |
| | | TCGATTTTTGTGATGCTCGTCAGGGGGGCGGAGCCTATGGAAAA |
| | | ACGCCAGCAACGCGGCCTTTTTACGGTTCCTGGCCTTTTGCTGGC |
| | | CTTTTGCTCACATGTTCTTTCCTGCGTTATCCCCTGATTCTGTGG |
| | | ATAACCGTATTACCGCCTTTGAGTGAGCTGATACCGCTCGCCGC |
| | | AGCCGAACGACCGAGCGCAGCGAGTCAGTGAGCGAGGAAGCGG |
| | | AAG |

1. An AAV capsid protein variant comprising an engineered capsid protein VP1, the VP1 comprising amino acid substitutions I240T and V708I relative to a parental AAV capsid protein VP1, wherein the amino acid positions are determined with reference to the position of amino acid sequence of SEQ ID NO: 1.

2. The AAV capsid protein variant according to claim 1, further comprising a Y444F substitution and/or a T491V substitution.

3. The AAV capsid protein variant according to claim 1 or 2, further comprising an insertion of 5-15 amino acids, such as 5-10 amino acids, preferably 10 amino acids, between positions 587 and 588.

4. The AAV capsid protein variant according to claim 3, wherein fragments LALGETTRPA, LALGDVTRPA, or LALGEVTRPA is inserted between positions 587 and 588.

5. The AAV capsid protein variant according to any one of claims 1 to 4, comprising, or consisting of only, the following amino acid mutations;
(1) I240T-V708I and an insertion of a fragment LALGETTRPA, LALGDVTRPA, or LALGEVTRPA between positions 587-588;
(2) I240T-V708I-Y444F and an insertion of a fragment LALGETTRPA, LALGDVTRPA, or LALGEVTRPA between positions 587-588;
(3) I240T-V708I-Y444F-T491V and an insertion of the fragments LALGETTRPA, LALGDVTRPA, or LALGEVTRPA between positions 587-588.

6. The AAV capsid protein variant according to claim 1 or 2, wherein the parental AAV capsid protein VP1 is from AAV serotype 2 (AAV2) or AAV2 variant version 7m8 (AAV2.7m8).

7. The AAV capsid protein variant according to any one of claims 3 to 5, wherein the parental AAV capsid protein VP1 is from AAV serotype 2 (AAV2), preferably the parental capsid protein VP1 comprises or consists of the amino acid sequence as set forth in SEQ ID NO:1.

8. The AAV capsid protein variant according to any one of claims 1 to 7, wherein the AAV capsid protein variant
(i) comprises the amino acid sequence as set forth in SEQ ID NO:2: or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:2, and comprises the substitutions I240T-V708I and the insert 587-LALGETTRPA-588;
(ii) comprises the amino acid sequence as set forth in SEQ ID NO:3: or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:3, and comprises the substitutions I240T-V708I-Y444F and the insert 587-LALGETTRPA-588;
(iii) comprises the amino acid sequence as set forth in SEQ ID NO:4; or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:4, and comprises the substitutions I240T-V708I and the insert 587-LALGEVTRPA-588; or
(iv) comprises the amino acid sequence as set forth in SEQ ID NO:5; or comprises an amino acid sequence having at least 85%, 86%, 87%, 88%, 89%, 90%, 91%, 92%, 93%, 94%, 95%, 96%, 97%, 98%, or 99% identity to the amino acid sequence as set forth in SEQ ID NO:5, and comprises the substitutions I240T-V708I and the insert 587-LALGDVTRPA-588.

9. The AAV capsid protein variant according to any one of claims 1 to 7, which is
(i) encoded by the nucleic acid sequence as set forth in SEQ ID NO:7; or encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:7 and comprises the substitution I240T-V708I and the insert 587-LALGETTRPA-588;
(ii) encoded by the nucleic acid sequence as set forth in SEQ ID NO:8: or encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:8 and comprises the substitution I240T-V708I-Y444F and the insert 587-LALGETTRPA-588;
(iii) encoded by the nucleic acid sequence as set forth in SEQ ID NO:9: or encoded by a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO:9 and comprises the substitution I240T-V708I and the insert 587-LALGEVTRPA-588; or
(iv) encoded by the nucleic acid sequence as set forth in SEQ ID NO:10; or encoded by a nucleic acid sequence having at least 85%, 86%, 87%, 88%, 89%, 90%, 91%, 92%, 93%, 94%, 95%, 96%, 97%, 98% or 99% identity to the nucleic acid sequence as set forth in SEQ ID NO:10 and comprises the substitution I240T-V708I and the insert 587-LALGDVTRPA-588.

10. An isolated nucleic acid comprising a nucleotide sequence encoding the AAV capsid protein variant according to any one claims 1 to 9.

11. The isolated nucleic acid according to claim 10, wherein the nucleotide sequence encodes a capsid protein, which
(i) comprises or consists of the nucleic acid sequence as set forth in SEQ ID NO:7; or comprises a nucleic acid sequence having at least 85%, 86%, 87%, 88%, 89%, 90%, 91%, 92%, 93%, 94%, 95%, 96%, 97%, 98% or 99% identity to the nucleic acid sequence as set forth in SEQ ID NO: 7, and the capsid protein variant encoded by said nucleic acid sequence comprises the substitution I240T-V708I and the insert 587-LALGETTRPA-588;
(ii) comprises or consists of the nucleic acid sequence as set forth in SEQ ID NO:8: or comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO: 8, and the capsid protein variant encoded by said nucleic acid sequence comprises the substitution I240T-V708I-Y444F and the insert 587-LALGETTRPA-588;
(iii) comprises or consists of the nucleic acid sequence as set forth in SEQ ID NO:9; or comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO: 9, and the capsid protein variant encoded by said nucleic acid sequence comprises the substitution I240T-V708I and the insert 587-LALGEVTRPA-588; or
(iv) comprises or consists of the nucleic acid sequence as set forth in SEQ ID NO:10; or comprises a nucleic acid sequence having at least 85%, 86%, 87%, 88%, 89%, 90%, 91%, 92%, 93%, 94%, 95%, 96%, 97%, 98%, or 99% identity to the nucleic acid sequence as set forth in SEQ ID NO: 10, and the capsid protein variant encoded by said nucleic acid sequence comprises the substitution I240T-V708I and the insert 587-LALGDVTRPA-588.

12. A recombinant AAV viral particle (rAAV) comprising
(i) the AAV capsid protein variant of any one of claims 1 to 9; and optionally
(ii) a nucleic acid of interest encoding, e.g., a prophylactic or therapeutic protein, packaged within the AAV capsid, e.g., the nucleic acid of interest is selected from an ophthalmic related gene, e.g., RPE65, AIPL1, PROM1, or RS1.

13. A recombinant AAV viral particle according to claim 12, wherein the nucleic acid of interest is comprised in an expression cassette and thus packaged within the AAV capsid.

14. The recombinant AAV viral particle according to claim 13, wherein the expression cassette is single-stranded DNA, double-stranded DNA or single-stranded RNA or double-stranded RNA.

15. A method of producing a recombinant AAV viral particle, comprising culturing a packaging cell under conditions sufficient for production of a recombinant AAV viral particle, wherein the packaging cell comprises a plasmid comprising a nucleic acid encoding the capsid protein variant of any one of claims 1-9 or the nucleic acid of claim 10 or 11.

16. The method according to claim 15, wherein the packaging cell further comprises a helper plasmid and/or a transfer plasmid comprising the nucleic acid of interest.

17. The method of claim 15 or 16, further comprising isolating from the culture supernatant a self-complementary recombinant adeno-associated virus (rcAAV) particle.

18. The method according to any one of claims 15 to 17, further comprising lysing the packaging cell, and isolating a recombinant AAV viral particle from the cell lysate.

19. The method according to any one of claims 15 to 18, further comprising:
a. removing the cell debris,
b. treating the supernatant containing the recombinant AAV viral particle with a benzonase nuclease,
c. concentrating the recombinant AAV viral particle, and
d. purifying the recombinant AAV viral particle.

20. A recombinant AAV viral particle prepared according to the method of any one of claims 15 to 19.

21. A plasmid, such as an expression plasmid, comprising a nucleic acid encoding the capsid protein variant of any one of claims 1-9 or the nucleic acid of claim 10 or 11.

22. A packaging cell for producing a recombinant AAV viral particle, the packaging cell comprising a plasmid comprising a nucleic acid encoding the capsid protein variant of any one of claims 1-9 or the nucleic acid of claim 10 or 11.

23. A formulation or composition or medicament comprising the recombinant AAV viral particle of any one claim 12-14 or 20, and optionally pharmaceutically acceptable auxiliary materials, such as pharmaceutically acceptable carriers, excipients, including buffers, as known in the art.

24. A combination product comprising the recombinant AAV viral particle according to any of claim 12-14 or 20, and one or more additional therapeutic agents, e.g., an immunomodulator, e.g., an immunosuppressant.

25. A method of treating an ocular disease in an individual comprising administering to the individual the recombinant AAV viral particle of any one of claim 12-14 or 20 or the formulation or composition of claim 23 or the combination product of claim 24.

26. The method according to claim 25, wherein the administration can be an intraocular administration, e.g., an intraretinal administration or intravitreal administration, e.g., a subretinal administration or intravitreal administration.

27. The method according to claim 26, wherein the administration is injection.
